SEQUENCE LISTING (309 sequences)

SEQ ID NO: 1 - protein, 667 aa, Homo sapiens
METLESELTCPICLELFEDPLLLPCAHSLCFNCAHRILVSHCATNESVESITAFQCPTCRHVITLSQRGLDGLKRNVTLQNIIDRFQKASVSGPNSPSETRRERAFDANTMTSAEKVLCQFCDQDPAQDAVKTCVTCEVSYCDECLKATHPNKKPFTGHRLIEPIPDSHIRGLMCLEHEDEKVNMYCVTDDQLICALCKLVGRHRDHQVAALSERYDKLKQNLESNLTNLIKRNTELETLLAKLIQTCQHVEVNASRQEAKLTEECDLLIEIIQQRRQIIGTKIKEGKVMRLRKLAQQIANCKQCIERSASLISQAEHSLKENDHARFLQTAKNITERVSMATASSQVLIPEINLNDTFDTFALDFSREKKLLECLDYLTAPNPPTIREELCTASYDTITVHWTSDDEFSVVSYELQYTIFTGQANVVSLCNSADSWMIVPNIKQNHYTVHGLQSGTKYIFMVKAINQAGSRSSEPGKLKTNSQPFKLDPKSAHRKLKVSHDNLTVERDESSSKKSHTPERFTSQGSYGVAGNVFIDSGRHYWEVVISGSTWYAIGLAYKSAPKHEWIGKNSASWALCRCNNNWVVRHNSKEIPIEPAPHLRRVGILLDYDNGSIAFYDALNSIHLYTFDVAFAQPVCPTFTVWNKCLTIITGLPIPDHLDCTEQLP

SEQ ID NO: 2 - DNA, 2004 bp, Homo sapiens
atggaaacactggagtcagaactgacctgccctatttgtctggagctctttgaggaccctcttctactgccctgcgcacacagcctctgcttcaactgcgcccaccgcatcctagtatcacactgtgccaccaacgagtctgtggagtccatcaccgccttccagtgccccacctgccggcatgtcatcaccctcagccagcgaggtctagacgggctcaagcgcaacgtcaccctacagaacatcatcgacaggttccagaaagcatcagtgagcgggcccaactctcccagcgagacccgtcgggagcgggcctttgacgccaacaccatgacctccgccgagaaggtcctctgccagttttgtgaccaggatcctgcccaggacgctgtgaagacctgtgtcacttgtgaagtatcctactgtgacgagtgcctgaaagccactcacccgaataagaagccctttacaggccatcgtctgattgagccaattccggactctcacatccgggggctgatgtgcttggagcatgaggatgagaaggtgaatatgtactgtgtgaccgatgaccagttaatctgtgccttgtgtaaactggttgggcggcaccgcgatcatcaggtggcagctttgagtgagcgctatgacaaattgaagcaaaacttagagagtaacctcaccaaccttattaagaggaacacagaactggagacccttttggctaaactcatccaaacctgtcaacatgttgaagtcaatgcatcacgtcaagaagccaaattgacagaggagtgtgatcttctcattgagatcattcagcaaagacgacagattattggaaccaagatcaaagaagggaaggtgatgaggcttcgcaaactggctcagcagattgcaaactgcaaacagtgcattgagcggtcagcatcactcatctcccaagcggaacactctctgaaggagaatgatcatgcgcgtttcctacagactgctaagaatatcaccgagagagtctccatggcaactgcatcctcccaggttctaattcctgaaatcaacctcaatgacacatttgacacctttgccttagatttttcccgagagaagaaactgctagaatgtctggattaccttacagctcccaaccctcccacaattagagaagagctctgcacagcttcatatgacaccatcactgtgcattggacctccgatgatgagttcagcgtggtctcctacgagctccagtacaccatattcaccggacaagccaacgtcgttagtctgtgtaattcggctgatagctggatgatagtacccaacatcaagcagaaccactacacggtgcacggtctgcagagcggcaccaagtacatcttcatggtcaaggccatcaaccaggcgggcagccgcagcagtgagcctgggaagttgaagacaaacagccaaccatttaaactggatcccaaatctgctcatcgaaaactgaaggtgtcccatgataacttgacagtagaacgtgatgagtcatcatccaagaagagtcacacacctgaacgcttcaccagccaggggagctatggagtagctggaaatgtgtttattgatagtggccggcattattgggaagtggtcataagtggaagcacatggtatgccattggtcttgcttacaaatcagccccgaagcatgaatggattgggaagaactctgcttcctgggcgctctgccgctgcaacaataactgggtggtgagacacaatagcaaggaaatccccattgagcctgccccccacctccggcgcgtgggcatcctgctggactatgataacggctctatcgccttttatgatgctttgaactccatccacctctacaccttcgacgtcgcatttgcgcagcctgtttgccccaccttcaccgtgtggaacaagtgtctgacgattatcactgggctccctatcccagaccatttggactgcacagagcagctgccgtga

SEQ ID NO: 3 - DNA, 21 bp, Artificial Sequence
  Synthetic siRNA sequence
uuagguaauccagacauucta

SEQ ID NO: 4 - DNA, 23 bp, Artificial Sequence
  Synthetic Oligonucleotide
acagtcagccgcatcttcttttg

SEQ ID NO: 5 - DNA, 23 bp, Artificial Sequence
  Synthetic Oligonucleotide
ccaatacgaccaaatccgttgac

SEQ ID NO: 6 - DNA, 23 bp, Artificial Sequence
  Synthetic Oligonucleotide
gtacaccatattcaccgacaagc

SEQ ID NO: 7 - DNA, 24 bp, Artificial Sequence
  Synthetic Oligonucleotide
agtggttctgcttgatgttgggta

SEQ ID NO: 8 - DNA, 22 bp, Artificial Sequence
  Synthetic Oligonucleotide
aggccacactttgttggatttg

SEQ ID NO: 9 - DNA, 24 bp, Artificial Sequence
  Synthetic Oligonucleotide
caacttgcgctcatcttaggcttt

SEQ ID NO: 10 - DNA, 23 bp, Artificial Sequence
  Synthetic Oligonucleotide
cactcgctgaaggaaaatgacca

SEQ ID NO: 11 - DNA, 24 bp, Artificial Sequence
  Synthetic Oligonucleotide
aatccaaggcaaaagtgtcaaacg

SEQ ID NO: 12 - RNA, 21 nt, Artificial Sequence
  Synthetic Oligonucleotide
uagacuauagagggacuucua

SEQ ID NO: 13 - RNA, 21 nt, Artificial Sequence
  Synthetic Oligonucleotide
aagguuggugagguuacucuc

SEQ ID NO: 14 - RNA, 21 nt, Artificial Sequence
  Synthetic Oligonucleotide
ugagauuucauuacacucaug

SEQ ID NO: 15 - RNA, 21 nt, Artificial Sequence
  Synthetic Oligonucleotide
ugaugcauugacuucaacaug

SEQ ID NO: 16 - RNA, 21 nt, Artificial Sequence
  Synthetic Oligonucleotide
gugauaaucgucagacacuug

SEQ ID NO: 17 - RNA, 21 nt, Artificial Sequence
  Synthetic Oligonucleotide
agaaccuuucuuuccaacauu

SEQ ID NO: 18 - RNA, 21 nt, Artificial Sequence
  Synthetic Oligonucleotide
cuguuugcaguuugcaaucug

SEQ ID NO: 19 - RNA, 21 nt, Artificial Sequence
  Synthetic Oligonucleotide
gugugugacucuucuuggaug

SEQ ID NO: 20 - RNA, 21 nt, Artificial Sequence
  Synthetic Oligonucleotide
ucagugacuuauguuuggaug

SEQ ID NO: 21 - RNA, 21 nt, Artificial Sequence
  Synthetic Oligonucleotide
cuaugagaagguaaagauuua

SEQ ID NO: 22 - RNA, 21 nt, Artificial Sequence
  Synthetic Oligonucleotide
caguaggauacuucacaagug

SEQ ID NO: 23 - RNA, 21 nt, Artificial Sequence
  Synthetic Oligonucleotide
aucacguucuacugucaaguu

SEQ ID NO: 24 - RNA, 21 nt, Artificial Sequence
  Synthetic Oligonucleotide
gaaucuagcaaauaguacuuu

SEQ ID NO: 25 - RNA, 21 nt, Artificial Sequence
  Synthetic Oligonucleotide
agaguucuucccaauccauuc

SEQ ID NO: 26 - RNA, 21 nt, Artificial Sequence
  Synthetic Oligonucleotide
ugugaugaaaugaagguugua

SEQ ID NO: 27 - RNA, 21 nt, Artificial Sequence
  Synthetic Oligonucleotide
ucugcuugauguuggguacua

SEQ ID NO: 28 - RNA, 21 nt, Artificial Sequence
  Synthetic Oligonucleotide
augcauugacuucaacauguu

SEQ ID NO: 29 - RNA, 21 nt, Artificial Sequence
  Synthetic Oligonucleotide
acuuaugggccucuuaaugug

SEQ ID NO: 30 - RNA, 21 nt, Artificial Sequence
  Synthetic Oligonucleotide
ucagccgaauuacacagacua

SEQ ID NO: 31 - RNA, 21 nt, Artificial Sequence
  Synthetic Oligonucleotide
guguuccucuuaauaagguug

SEQ ID NO: 32 - RNA, 21 nt, Artificial Sequence
  Synthetic Oligonucleotide
aggaagcugaucagcuaucug

SEQ ID NO: 33 - RNA, 21 nt, Artificial Sequence
  Synthetic Oligonucleotide
gcuuccacuuaugaccacuuc

SEQ ID NO: 34 - RNA, 21 nt, Artificial Sequence
  Synthetic Oligonucleotide
ucggugauauucuuagcaguc

SEQ ID NO: 35 - RNA, 21 nt, Artificial Sequence
  Synthetic Oligonucleotide
uagagggacuucuacaucuuu

SEQ ID NO: 36 - RNA, 21 nt, Artificial Sequence
  Synthetic Oligonucleotide
accuaccaugggaucaucaug

SEQ ID NO: 37 - RNA, 21 nt, Artificial Sequence
  Synthetic Oligonucleotide
agaccaauggcauaccaugug

SEQ ID NO: 38 - RNA, 21 nt, Artificial Sequence
  Synthetic Oligonucleotide
gaaccugucgaugauguucug

SEQ ID NO: 39 - RNA, 21 nt, Artificial Sequence
  Synthetic Oligonucleotide
ugugacucuucuuggaugaug

SEQ ID NO: 40 - RNA, 21 nt, Artificial Sequence
  Synthetic Oligonucleotide
guugggagcuguaagguaauc

SEQ ID NO: 41 - RNA, 21 nt, Artificial Sequence
  Synthetic Oligonucleotide
gaucacacuccucugucaauu

SEQ ID NO: 42 - RNA, 21 nt, Artificial Sequence
  Synthetic Oligonucleotide
acucaugaagccugugcuuuc

SEQ ID NO: 43 - RNA, 21 nt, Artificial Sequence
  Synthetic Oligonucleotide
aaacuuaugggccucuuaaug

SEQ ID NO: 44 - RNA, 21 nt, Artificial Sequence
  Synthetic Oligonucleotide
uaggaaacgcgcaugaucauu

SEQ ID NO: 45 - RNA, 21 nt, Artificial Sequence
  Synthetic Oligonucleotide
guugacagguuuggaugaguu

SEQ ID NO: 46 - RNA, 21 nt, Artificial Sequence
  Syhthetic Oligonucleotide
gcacagugugauacuaggaug

SEQ ID NO: 47 - RNA, 21 nt, Artificial Sequence
  Synthetic Oligonucleotide
guucaaacacauccuguuaug

SEQ ID NO: 48 - RNA, 21 nt, Artificial Sequence
  Synthetic Oligonucleotide
aaaguggugcugcuauuugug

SEQ ID NO: 49 - RNA, 21 nt, Artificial Sequence
  Synthetic Oligonucleotide
acguucuacugucaaguuauc

SEQ ID NO: 50 - RNA, 21 nt, Artificial Sequence
  Synthetic Oligonucleotide
gauggaguucaaagcaucaua

SEQ ID NO: 51 - RNA, 21 nt, Artificial Sequence
  Synthetic Oligonucleotide
guagugguucugcuugauguu

SEQ ID NO: 52 - RNA, 21 nt, Artificial Sequence
  Synthetic Oligonucleotide
aucacacuccucugucaauuu

SEQ ID NO: 53 - RNA, 21 nt, Artificial Sequence
  Synthetic Oligonucleotide
guguagugguucugcuugaug

SEQ ID NO: 54 - RNA, 21 nt, Artificial Sequence
  Synthetic Oligonucleotide
ugcuauuugugacagaacaug

SEQ ID NO: 55 - RNA, 21 nt, Artificial Sequence
  Syunthetic Oligonucleotide
acucgucacaguaggauacuu

SEQ ID NO: 56 - RNA, 21 nt, Artificial Sequence
  Synthetic Oligonucleotide
ggaugaugacucaucacguuc

SEQ ID NO: 57 - RNA, 21 nt, Artificial Sequence
  Synthetic Oligonucleotide
ugcauugacuucaacauguug

SEQ ID NO: 58 - RNA, 21 nt, Artificial Sequence
  Synthetioc Oligonucleotide
auagggagcccagugauaauc

SEQ ID NO: 59 - RNA, 21 nt, Artificial Sequence
  Synthetic Oligonucleotide
aucaccuucccuucuuugauc

SEQ ID NO: 60 - RNA, 21 nt, Artificial Sequence
  Synthetic Oligonucleotide
uggaaccugucgaugauguuc

SEQ ID NO: 61 - RNA, 21 nt, Artificial Sequence
  Synthetic Oligonucleotide
ugcuugauguuggguacuauc

SEQ ID NO: 62 - RNA, 21 nt, Artificial Sequence
  Synthetic Oligonucleotide
accugucgaugauguucugua

SEQ ID NO: 63 - RNA, 21 nt, Artificial Sequence
  Synthetic Oligonucleotide
uucccaauccauucaugcuuc

SEQ ID NO: 64 - RNA, 21 nt, Artificial Sequence
  Synthetic Oligonucleotide
guuguaauccugauuauguuu

SEQ ID NO: 65 - RNA, 21 nt, Artificial Sequence
  Synthetic Oligonucleotide
augggaucaucaugggauauc

SEQ ID NO: 66 - RNA, 21 nt, Artificial Sequence
  Synthetic Oligonucleotide
gguaagaagaugaguaacaua

SEQ ID NO: 67 - RNA, 21 nt, Artificial Sequence
  Synthetic Oligonucleotide
ugaucagcuaucugccucaug

SEQ ID NO: 68 - RNA, 21 nt, Artificial Sequence
  Synthetic Oligonucleotide
agaagaucacacuccucuguc

SEQ ID NO: 69 - RNA, 21 nt, Artificial sequence
  Synthetic Oligonucleotide
cuucaacauguugacagguuu

SEQ ID NO: 70 - RNA, 21 nt, Artificial Sequence
  Synthetic Oligonucleotide
ccaacauuaucccaucagcua

SEQ ID NO: 71 - RNA, 21 nt, Artificial Sequence
  Synthetic Oligonucleotide
acuagguucaaacacauccug

SEQ ID NO: 72 - RNA, 21 nt, Artificial Sequence
  Synthetic Oligonucleotide
caagaccaauggcauaccaug

SEQ ID NO: 73 - RNA, 21 nt, Artificial Sequence
  Synthetic Oligonucleotide
acuagacuauagagggacuuc

SEQ ID NO: 74 - RNA, 21 nt, Artificial Sequence
  Synthetic Oligonucleotide
auggugucauaugaagcugug

SEQ ID NO: 75 - RNA, 21 nt, Artificial Sequence
  Synthetic Oligonucleotide
uucaacauguugacagguuug

SEQ ID NO: 76 - RNA, 21 nt, Artificial Sequence
  Synthetic Oligonucleotuide
gauugugaugaaaugaagguu

SEQ ID NO: 77 - RNA, 21 nt, Artificial Sequence
  Synthetic Oligonucleotide
cagauuaacuggucaucgguc

SEQ ID NO: 78 - RNA, 21 nt, Artificial Sequence
  Synthetic Oligonucleotide
auagagggacuucuacaucuu

SEQ ID NO: 79 - RNA, 21 nt, Artificial Sequence
  Synthetic Oligonucleotide
aaugguuggcuguuugucuuc

SEQ ID NO: 80 - RNA, 21 nt, Artificial Sequence
  Synthetic Oligonucleotide
cuguaagguaauccagacauu

SEQ ID NO: 81 - RNA, 21 nt, Artificial Sequence
  Synthetic Oligonucleotide
gcaaagcucucuugugucauc

SEQ ID NO: 82 - RNA, 21 nt, Artificial Sequence
  Synthetic Oligonucleotide
guuuacacaaggcacagauua

SEQ ID NO: 83 - RNA, 21 nt, Artificial Sequence
  Synthetic Oligonucleotide
uuuccagcuacuccauagcuc

SEQ ID NO: 84 - RNA, 21 nt, Artificial Sequence
  Synthetic Oligonucleotide
cuucuuggaugaugacucauc

SEQ ID NO: 85 - RNA, 21 nt, Artificial Sequence
  Synthetic Oligonucleotide
aagaccuaccaugggaucauc

SEQ ID NO: 86 - RNA, 21 nt, Artificial Sequence
  Synthetic Oligonucleotide
uugacagguuuggaugaguuu

SEQ ID NO: 87 - RNA, 21 nt, Artificial Sequence
  Synthetic Oligonucleotide
auuugugacagaacauguuua

SEQ ID NO: 88 - RNA, 21 nt, Artificial Sequence
  Synthetic Oligonucleotide
agugauaaucgucagacacuu

SEQ ID NO: 89 - RNA, 21 nt, Artificial Sequence
  Synthetic Oligonucleotide
gauaaucgucagacacuuguu

SEQ ID NO: 90 - RNA, 21 nt, Artificial Sequence
  Synthetic Oligonucleotide
gaagcagaguucuucccaauc

SEQ ID NO: 91 - RNA, 21 nt, Artificial Sequence
  Synthetic Oligonucleotide
aucagcuaucugccucauguu

SEQ ID NO: 92 - RNA, 21 nt, Artificial Sequence
  Synthetic Oligonucleotide
agguucaaacacauccuguua

SEQ ID NO: 93 - RNA, 21 nt, Artificial Sequence
  Synthetic Oligonucleotide
uguaagguaauccagacauuc

SEQ ID NO: 94 - RNA, 21 nt, Artificial Sequence
  Synthetic Oligonucleotide
uugacgugaugcauugacuuc

SEQ ID NO: 95 - RNA, 21 nt, Artificial Sequence
  Synthetic Oligonucleotide
uccaguuuaaaugguuggcug

SEQ ID NO: 96 - RNA, 21 nt, Artificial Sequence
  Syhthetic Oligonucleotide
uauuaaagcccguacuuaaua

SEQ ID NO: 97 - RNA, 21 nt, Artificial Sequence
  Synthetic Oligonucleotide
gaaacgcgcaugaucauucuc

SEQ ID NO: 98 - RNA, 21 nt, Artificial Sequence
  Synthetic Oligonucleotide
guacauauucaccuucucauc

SEQ ID NO: 99 - RNA, 21 nt, Artificial Sequence
  Synthetic Oligonucleotide
auacaaugucccuugucauua

SEQ ID NO: 100 - RNA, 21 nt, Artificial Sequence
  Synthetic Oligonucleotide
cagaguucuucccaauccauu

SEQ ID NO: 101 - RNA, 21 nt, Artificial Sequence
  Synthetic Oligonucleotide
uucuacugucaaguuaucaug

SEQ ID NO: 102 - RNA, 21 nt, Artificial Sequence
  Synthetic Oligonucleotide
cuugacgugaugcauugacuu

SEQ ID NO: 103 - RNA, 21 nt, Artificial Sequence
  Synthetic Oligonucleotide
uugauguuggguacuaucauc

SEQ ID NO: 104 - RNA, 21 nt, Artificial Sequence
  Synthetic Oligonucleotide
uucuggaaccugucgaugaug

SEQ ID NO: 105 - RNA, 21 nt, Artificial Sequence
  Synthetic Oligonucleotide
uggaugaugacucaucacguu

SEQ ID NO: 106 - RNA, 21 nt, Artificial Sequence
  Synthetic Oligonucleotide
uaguugaucugauacaacuuu

SEQ ID NO: 107 - RNA, 21 nt, Artificial Sequence
  Synthetic Oligonucleotide
ucaucggucacacaguacaua

SEQ ID NO: 108 - RNA, 21 nt, Artificial Sequence
  Synthetic Oligonucleotide
ucuugguuccaauaaucuguc

SEQ ID NO: 109 - RNA, 21 nt, Artificial Sequence
  Synthetic Oligonucleotide
uuccucuuaauaagguuggug

SEQ ID NO: 110 - RNA, 21 nt, Artificial Sequence
  Synthetic Oligonucleotide
ggaaggauaaaguagauucua

SEQ ID NO: 111 - RNA, 21 nt, Artificial Sequence
  Synthetic Oligonucleotide
ugacagguuuggaugaguuua

SEQ ID NO: 112 - RNA, 21 nt, Artificial Sequence
  Synthetic Oligonucleotide
ucacguucuacugucaaguua

SEQ ID NO: 113 - RNA, 21 nt, Artificial Sequence
  Synthetic Oligonucleotide
acucucucggugauauucuua

SEQ ID NO: 114 - RNA, 21 nt, Artificial Sequence
  Synthetic Oligonucleotide
ggguacuaucauccagcuauc

SEQ ID NO: 115 - RNA, 21 nt, Artificial Sequence
  Synthetic Oligonucleotide
gacucaucacguucuacuguc

SEQ ID NO: 116 - RNA, 21 nt, Artificial Sequence
  Synthetic oligonucleotide
uaccaugugcuuccacuuaug

SEQ ID NO: 117 - RNA, 21 nt, Artificial Sequence
  Synthetic Oligonucleotide
cacaggucuaaaggaacuauu

SEQ ID NO: 118 - RNA, 21 nt, Artificial Sequence
  Synthetic Oligonucleotide
ccacuaucaauaaacacauuu

SEQ ID NO: 119 - RNA, 21 nt, Artificial Sequence
  Synthetic Oligonucleotide
uugaucuugguuccaauaauc

SEQ ID NO: 120 - RNA, 21 nt, Artificial Sequence
  Synthetic Oligonucleotide
aucggucacacaguacauauu

SEQ ID NO: 121 - RNA, 21 nt, Artificial Sequence
  Synthetic Oligonucleotide
aggaaacgcgcaugaucauuc

SEQ ID NO: 122 - RNA, 21 nt, Artificial Sequence
  Synthetic Oligonucleotide
gccacuaucaauaaacacauu

SEQ ID NO: 123 - RNA, 21 nt, Artificial Sequence
  Synthetic Oligonucleotide
aggauucagaaccuuucuuuc

SEQ ID NO: 124 - RNA, 21 nt, Artificial Sequence
  Synthetic Oligonucleotide
uccggugaauaugguguacug

SEQ ID NO: 125 - RNA, 21 nt, Artificial Sequence
  Synthetic Oligunucleotide
gaucuugguuccaauaaucug

SEQ ID NO: 126 - RNA, 21 nt, Artificial Sequence
  Synthetic Oligonucleotide
ggcaaaggugucaaauguguc

SEQ ID NO: 127 - RNA, 21 nt, Artificial Sequence
  Synthetic Oligonucleotide
auccagcuaucagccgaauua

SEQ ID NO: 128 - RNA, 21 nt, Artificial Sequence
  Synthetic Oligonucleotide
ugucgucuuugcugaaugauc

SEQ ID NO: 129 - RNA, 21 nt, Artificial Sequence
  Synthetic Oligonucleotide
gcaaucucauucuuaauguuc

SEQ ID NO: 130 - RNA, 21 nt, Artificial Sequence
  Synthetic Oligonucleotide
cucgucacaguaggauacuuc

SEQ ID NO: 131 - RNA, 21 nt, Artificial Sequence
  Synthetic Oligonucleotide
gugagguuacucucuaaguuu

SEQ ID NO: 132 - RNA, 21 nt, Artificial Sequence
  Synthetic Oligonucleotide
uccagacauucuagcaguuuc

SEQ ID NO: 133 - RNA, 21 nt, Artificial Sequence
  Synthetic Oligonucleotide
gggauccaguuuaaaugguug

SEQ ID NO: 134 - RNA, 21 nt, Artificial Sequence
  Synthetic Oligonucleotide
ucagcuaucugccucauguuu

SEQ ID NO: 135 - RNA, 21 nt, Artificial Sequence
  Synthetic Oligonucleotide
agacauucuagcaguuucuuc

SEQ ID NO: 136 - RNA, 21 nt, Artificial Sequence
  Synthetic Oligonucleotide
ucaggugugugacucuucuug

SEQ ID NO: 137 - RNA, 21 nt, Artificial Sequence
  Synthetic Oligonucleotide
gacucucucggugauauucuu

SEQ ID NO: 138 - RNA, 21 nt, Artificial Sequence
  Synthetic Oligonucleotide
cagagcucuucucuaauugug

SEQ ID NO: 139 - RNA, 21 nt, Artificial Sequence
  Synthetic Oligonucleotide
ugcaaucucauucuuaauguu

SEQ ID NO: 140 - RNA, 21 nt, Artificial Sequence
  Synthetic Oligonucleotide
gggauaucagguaagaagaug

SEQ ID NO: 141 - RNA, 21 nt, Artificial Sequence
  Synthetic Oligonucleotide
acaggucuaaaggaacuauua

SEQ ID NO: 142 - RNA, 21 nt, Artificial Sequence
  Synthetic Oligonucleotide
gagacucucucggugauauuc

SEQ ID NO: 143 - RNA, 21 nt, Artificial Sequence
  Synthetic oligonucleotide
ugggauccaguuuaaaugguu

SEQ ID NO: 144 - RNA, 21 nt, Artificial Sequence
  Synthetic Oligonucleotide
uuggcuuguccggugaauaug

SEQ ID NO: 145 - RNA, 21 nt, Artificial Sequence
  Synthetic Oligonucleotide
ucacacuccucugucaauuug

SEQ ID NO: 146 - RNA, 21 nt, Artificial Sequence
  Synthetic Oligonucleotide
gccuguaaagggcuucuuauu

SEQ ID NO: 147 - RNA, 21 nt, Artificial Sequence
  Synthetic Oligonucleotide
ccuguaaagggcuucuuauuc

SEQ ID NO: 148 - RNA, 21 nt, Artificial Sequence
  Synthetic Oligonucleotide
gucauuaccaacucauaaaua

SEQ ID NO: 149 - RNA, 21 nt, Artificial Sequence
  Synthetic Oligonucleotide
aaggcaaaggugucaaaugug

SEQ ID NO: 150 - RNA, 21 nt, Artificial Sequence
  Synthetic Oligonucleotide
ggugagguuacucucuaaguu

SEQ ID NO: 151 - RNA, 21 nt, Artificial Sequence
  Synthetic Oligonucleotide
uggucuuacaacucuauugua

SEQ ID NO: 152 - RNA, 21 nt, Artificial Sequence
  Synthetic Oligonucleotide
gaggcaugaaucuagcaaaua

SEQ ID NO: 153 - RNA, 21 nt, Artificial Sequence
  Synthetic Oligonucleotide
ugaucauucuccuucagaguu

SEQ ID NO: 154 - RNA, 21 nt, Artificial Sequence
  Synthetic Oligonucleotide
ugugcuuccacuuaugaccuu

SEQ ID NO: 155 - RNA, 21 nt, Artificial Sequence
  Synthetic Oligonucleotide
acagugugauacuaggauguu

SEQ ID NO: 156 - RNA, 21 nt, Artificial Sequence
  Synthetic Oligonucleotide
uugacuucaacauguugacuu

SEQ ID NO: 157 - RNA, 21 nt, Artificial Sequence
  Synthetic oligonucleotide
ugaucucaaugagaagaucuu

SEQ ID NO: 158 - RNA, 21 nt, Artificial Sequence
  Synthetic Oligonucleotide
acaaggcacagauuaacuguu

SEQ ID NO: 159 - RNA, 21 nt, Artificial Sequence
  Synthetic Oligonucleotide
augugguagugcugaucaguu

SEQ ID NO: 160 - RNA, 21 nt, Artificial Sequence
  Synthetic Oligonucleotide
uccaguuuaaaugguuggcuu

SEQ ID NO: 161 - RNA, 21 nt, Artificial Sequence
  Synthetic oligonucloetide
ucagacacuuguuccacacuu

SEQ ID NO: 162 - RNA, 21 nt, Artificial Sequence
  Synthetic Oligonucleotide
agguuuggaugaguuuagcuu

SEQ ID NO: 163 - RNA, 21 nt, Artificial Sequence
  Synthetic Oligonucleotide
auagcugauaaacuuaugguu

SEQ ID NO: 164 - RNA, 21 nt, Artificial Sequence
  Synthetic Oligonucleotide
uggaugaugacucaucacguu

SEQ ID NO: 165 - RNA, 21 nt, Artificial Sequence
  Synthetic Oligonucleotide
auccaguuuaaaugguugguu

SEQ ID NO: 166 - RNA, 21 nt, Artificial Sequence
  Synthetic Oligonucleotide
aucacguucuacugucaaguu

SEQ ID NO: 167 - RNA, 21 nt, Artificial Sequence
  Synthetic Oligonucleotide
accacccaguuauuguugcuu

SEQ ID NO: 168 - RNA, 21 nt, Artificial Sequence
  Synthetic Oligonucleotide
uuuguaagcaagaccaauguu

SEQ ID NO: 169 - RNA, 21 nt, Artificial Sequence
  Synthteic Oligonucleotide
ugcuucaauuugucauagcuu

SEQ ID NO: 170 - RNA, 21 nt, Artificial Sequence
  Synthetic oligonucleotide
uuccucuuaauaagguugguu

SEQ ID NO: 171 - RNA, 21 nt, Artificial Sequence
  Synthetic Oligonucleotide
uucuugacgugaugcauuguu

SEQ ID NO: 172 - RNA, 21 nt, Artificial Sequence
  SYnthetic Oligonucleotide
uggugcugcuauuugugacuu

SEQ ID NO: 173 - RNA, 21 nt, Artificial Sequence
  Synthetic Oligonucleotide
uucuuccagaacccuuaacuu

SEQ ID NO: 174 - RNA, 21 nt, Artificial Sequence
  Synthetic Oligonucleotide
ugugaugaaaugaagguuguu

SEQ ID NO: 175 - RNA, 21 nt, Artificial Sequence
  Synthetic Oligonucleotide
ucugcuugauguuggguacuu

SEQ ID NO: 176 - RNA, 21 nt, Artificial Sequence
  Synthetic Oligonucleotuide
uaccaugggaucaucaugguu

SEQ ID NO: 177 - RNA, 21 nt, Artificial Sequence
  Synthetic Oligonucleotide
ucggugauauucuuagcaguu

SEQ ID NO: 178 - RNA, 21 nt, Artificial Sequence
  Synthetic Oligonucleotide
aggaagcugaucagcuaucuu

SEQ ID NO: 179 - RNA, 21 nt, Artificial Sequence
  Syhthetic Oligonucleotide
acacagacuaacgacguuguu

SEQ ID NO: 180 - RNA, 21 nt, Artificial Sequence
  Synthetic Oligonucleotide
agcaagaccaauggcauacuu

SEQ ID NO: 181 - RNA, 21 nt, Artificial Sequence
  Synthetic Oligonucleotide
ucagccgaauuacacagacuu

SEQ ID NO: 182 - RNA, 21 nt, Artificial Sequence
  Synthetic Oligonucloetide
ucucggugauauucuuagcuu

SEQ ID NO: 183 - RNA, 21 nt, Artificial Sequence
  Synthetic Oligonucleotide
uuagcagucuguaggaaacuu

SEQ ID NO: 184 - RNA, 21 nt, Artificial Sequence
  Synthtetic Oligonucleotide
ucauagcgcucacucaaaguu

SEQ ID NO: 185 - RNA, 21 nt, Artificial Sequence
  Synthetic Oligonucleotide
acucgucacaguaggauacuu

SEQ ID NO: 186 - RNA, 21 nt, Artificial Sequence
  Synthetic Oligonucleotide
uccaacauuaucccaucaguu

SEQ ID NO: 187 - RNA, 21 nt, Artificial Sequence
  Synthetic Oligonucleotide
ugacucuucuuggaugauguu

SEQ ID NO: 188 - RNA, 21 nt, Artificial Sequence
  Synthetic Oligonucleotide
ugacuuauguuuggauguguu

SEQ ID NO: 189 - RNA, 21 nt, Artificial Sequence
  Synthetic Oligonucleotide
ugucgaugauguucuguaguu

SEQ ID NO: 190 - RNA, 21 nt, Artificial Sequence
  Synthetic Oligonucleotide
aggugugugacucuucuuguu

SEQ ID NO: 191 - RNA, 21 nt, Artificial Sequence
  Synthetic Oligonucleotide
accugucgaugauguucuguu

SEQ ID NO: 192 - RNA, 21 nt, Artificial Sequence
  Synthetic Oligonucleotide
uccaaauggucugggauaguu

SEQ ID NO: 193 - RNA, 21 nt, Artificial Sequence
  Synthetic Oligonucleotide
uuggcucaaucagacgauguu

SEQ ID NO: 194 - RNA, 21 nt, Artificial Sequence
  Synthetic Oligonucleotide
uuggcuucuugacgugauguu

SEQ ID NO: 195 - RNA, 21 nt, Artificial Sequence
  Synthetic Oligonucleotide
augugagaguccggaauuguu

SEQ ID NO: 196 - RNA, 21 nt, Artificial Sequence
  Synthetic Oligonucleotide
uccggugaauaugguguacuu

SEQ ID NO: 197 - RNA, 21 nt, Artificial Sequence
  Synthetic Oligonucloetide
ugcaaucucauucuuaauguu

SEQ ID NO: 198 - RNA, 21 nt, Artificial Sequence
  Synthetic Oligonucleotide
acacuccucugucaauuuguu

SEQ ID NO: 199 - RNA, 21 nt, Artificial Sequence
  Synthetic Oligonucleotide
ugggauaucagguaagaaguu

SEQ ID NO: 200 - RNA, 21 nt, Artificial Sequence
  Synthetic Oligonucleotide
ugagaagguaaagauuuaguu

SEQ ID NO: 201 - RNA, 21 nt, Artificial Sequence
  Synthetic Oligonucleotide
ucuggugagauuucauuacuu

SEQ ID NO: 202 - RNA, 21 nt, Artificial Sequence
  Synthetic Oligonucleotide
agcccguacuuaauacaaguu

SEQ ID NO: 203 - RNA, 21 nt, Artificial Sequence
  Synthetic Oligonucleotide
uugggauccaguuuaaauguu

SEQ ID NO: 204 - RNA, 21 nt, Artificial Sequence
  Synthetic Oligonucleotide
uggucuuacaacucuauuguu

SEQ ID NO: 205 - RNA, 21 nt, Artificial Sequence
  Synthetic Oligonucleotide
ugggagcuguaagguaaucuu

SEQ ID NO: 206 - RNA, 21 nt, Artificial Sequence
  Synthetic Oligonucleotide
auuacacucaugaagccuguu

SEQ ID NO: 207 - RNA, 21 nt, Artificial Sequence
  Synthetic Oligonucleotide
augaagauguacuuggugcuu

SEQ ID NO: 208 - RNA, 21 nt, Artificial Sequence
  Synthetic Oligonucleotide
ugauaaacuuaugggccucuu

SEQ ID NO: 209 - RNA, 21 nt, Artificial Sequence
  Synthetic Oligonucleotide
uaauguucuuccagaacccuu

SEQ ID NO: 210 - RNA, 21 nt, Artificial Sequence
  Synthetic Oligonulcetide
uacuagacuauagagggacuu

SEQ ID NO: 211 - RNA, 21 nt, Artificial Sequence
  Synthetic Oligonucleotide
uaaacacauuuccagcuacuu

SEQ ID NO: 212 - RNA, 21 nt, Artificial Sequence
  Synthetic Oligonucleotide
auuacacagacuaacgacguu

SEQ ID NO: 213 - RNA, 21 nt, Artificial Sequence
  Synthetic Oligonucleotide
uugaucuguugcuacacgcuu

SEQ ID NO: 214 - RNA, 21 nt, Artificial Sequence
  Synthetic Oligonucleotide
agaagguaaagauuuagccuu

SEQ ID NO: 215 - RNA, 21 nt, Artificial Sequence
  Synthetic Oligonucleotide
ucauuacacucaugaagccuu

SEQ ID NO: 216 - RNA, 21 nt, Artificial Sequence
  Synthetic Oligonulceotide
uaaagggcuucuuauucgguu

SEQ ID NO: 217 - RNA, 21 nt, Artificial Sequence
  Synthetic Oligonucleotide
agcugauaaacuuaugggcuu

SEQ ID NO: 218 - RNA, 21 nt, Artificial Sequence
  Synthetic Oligonucleotide
ugaauaugguguacuggaguu

SEQ ID NO: 219 - RNA, 21 nt, Artificial Sequence
  Synthetic Oligonucleotide
aguacuuuauacaauguccuu

SEQ ID NO: 220 - RNA, 21 nt, Artificial Sequence
  Synthetic Oligonucleotide
uuugaggcaugaaucuagcuu

SEQ ID NO: 221 - RNA, 21 nt, Artificial Sequence
  Synthetic Oligonucleotide
uuugcaguuugcaaucugcuu

SEQ ID NO: 222 - RNA, 21 nt, Artificial Sequence
  Synthetic Oligonucleotide
ucaauccaaaguggugcuguu

SEQ ID NO: 223 - RNA, 21 nt, Artificial Sequence
  Synthetic Oligonucleotide
ugucaaguuaucaugggacuu

SEQ ID NO: 224 - RNA, 21 nt, Artificial Sequence
  Synthetic Oligonucleotide
auucuuagcagucuguagguu

SEQ ID NO: 225 - RNA, 21 nt, Artificial Sequence
  Synthetic Oligonucleotide
uagaaucccaugacccaucuu

SEQ ID NO: 226 - RNA, 21 nt, Artificial Sequence
  Synthetic Oligonucleotide
uuugaucuguugcuacacguu

SEQ ID NO: 227 - RNA, 21 nt, Artificial Sequence
  Synthetic Oligonucleotide
acuucaacauguugacagguu

SEQ ID NO: 228 - RNA, 21 nt, Artificial Sequence
  Synthetic Oligonucleotide
ugaagguuguaaauagcuguu

SEQ ID NO: 229 - RNA, 21 nt, Artificial Sequence
  Synthetic Oligonucleotide
uuaaaccagaguaagaaaguu

SEQ ID NO: 230 - RNA, 21 nt, Artificial Sequence
  Synthetic Oligonucleotide
acuagguucaaacacauccuu

SEQ ID NO: 231 - RNA, 21 nt, Artificial Sequence
  Synthetic Oligonucleotide
uuggguacuaucauccagcuu

SEQ ID NO: 232 - RNA, 21 nt, Artificial Sequence
  Synthetic Oligonucleotide
ucuucuuggaugaugacucuu

SEQ ID NO: 233 - RNA, 21 nt, Artificial sequence
  Synthetic Oligonucleotide
ugcuuccacuuaugaccacuu

SEQ ID NO: 234 - RNA, 21 nt, Artificial Sequence
  Synthetic Oligonucleotide
auuuccagcuacuccauaguu

SEQ ID NO: 235 - RNA, 21 nt, Artificial Sequence
  Synthetic Oligonucleotide
agauuucauuacacucauguu

SEQ ID NO: 236 - RNA, 21 nt, Artificial Sequence
  Synthetic Oligonucleotode
agguaaagauuuagccacguu

SEQ ID NO: 237 - RNA, 21 nt, Artificial Sequence
  Synthetic Oligonucleotide
auggugucauaugaagcuguu

SEQ ID NO: 238 - RNA, 21 nt, Artificial Sequence
  Synthetic Oligonucleotide
acugucaaguuaucauggguu

SEQ ID NO: 239 - RNA, 21 nt, Artificial Sequence
  Synthetic Oligonulceotide
acaagugacacaggucuucuu

SEQ ID NO: 240 - RNA, 21 nt, Artificial Sequence
  Synthetic Oligonucleotide
uuucauuacacucaugaaguu

SEQ ID NO: 241 - RNA, 21 nt, Artificial Sequence
  Synthetic Oligonucleotide
agagccguuaucauaguccuu

SEQ ID NO: 242 - RNA, 21 nt, Artificial Sequence
  Synthetic Oligonucleotide
uuuccagcuacuccauagcuu

SEQ ID NO: 243 - RNA, 21 nt, Artificial Sequence
  Synthetic Oligonucleotide
uuccuugcuauugugucucuu

SEQ ID NO: 244 - RNA, 21 nt, Artificial Sequence
  Synthetic Oligonucleotide
uaaagcccguacuuaauacuu

SEQ ID NO: 245 - RNA, 21 nt, Artificial Sequence
  Synthetic Oligonucleotide
aguacauauucaccuucucuu

SEQ ID NO: 246 - RNA, 21 nt, Artificial Sequence
  Synthetic Oligonucleotide
uagagccguuaucauagucuu

SEQ ID NO: 247 - RNA, 21 nt, Artificial Sequence
  Synthetic Oligonucleotide
uagcugauaaacuuauggguu

SEQ ID NO: 248 - RNA, 21 nt, Artificial Sequence
  Synthetic Oligonucleotide
uuguaagcaagaccaaugguu

SEQ ID NO: 249 - RNA, 21 nt, Artificial Sequence
  Synthetic Oligonucleotide
uuuagaaucccaugacccauc

SEQ ID NO: 250 - RNA, 21 nt, Artificial Sequence
  Synthetic Oligonucleotide
aauuugucauagcgcucacuc

SEQ ID NO: 251 - RNA, 21 nt, Artificial Sequence
  Synthetic Oligonucleotide
aaauuauuaaagcccguacuu

SEQ ID NO: 252 - RNA, 21 nt, Artificial Sequence
  Synthetic Oligonucleotide
auuacacucaugaagccugug

SEQ ID NO: 253 - RNA, 21 nt, Artificial Sequence
  Synthetic Oligonucleotide
uuaauaagguuggugagguua

SEQ ID NO: 254 - RNA, 21 nt, Artificial Sequence
  Synthetic Oligonucleotide
gaauaugguguacuggagcuc

SEQ ID NO: 255 - RNA, 21 nt, Artificial Sequence
  Synthetic Oligonucleotide
aauuggcucaaucagacgaug

SEQ ID NO: 256 - RNA, 21 nt, Artificial Sequence
  Synthetic Oligonucleotide
auuuccuugcuauugugucuc

SEQ ID NO: 257 - RNA, 21 nt, Artificial Sequence
  Synthetic Oligonucleotide
uacuagacuauagagggacuu

SEQ ID NO: 258 - RNA, 21 nt, Artificial Sequence
  Synthetic Oligonucleotide
uuauucgggugaguggcuuuc

SEQ ID NO: 259 - RNA, 21 nt, Artificial Sequence
  Synthetic Oligonucleotide
cuucaauuugucauagcgcuc

SEQ ID NO: 260 - RNA, 21 nt, Artificial Sequence
  Synthetic Oligonucleotide
ugauaaacuuaugggccucuu

SEQ ID NO: 261 - RNA, 21 nt, Artificial
  Synthetic Oligonucleotide
uaauguucuuccagaacccuu

SEQ ID NO: 262 - RNA, 21 nt, Artificial Sequence
  Synthetic Oligonucelotide
gaauuagaaccugggaggaug

SEQ ID NO: 263 - RNA, 21 nt, Artificial Sequence
  Synthetic Oligonucleotide
auuuggcuucuugacgugaug

SEQ ID NO: 264 - RNA, 21 nt, Artificial Sequence
  Synthetic Oligonucleotide
uaaacacauuuccagcuacuc

SEQ ID NO: 265 - RNA, 21 nt, Artificial Sequence
  Synthetic Oligonucleotide
auuacacagacuaacgacguu

SEQ ID NO: 266 - RNA, 21 nt, Artificial Sequence
  Synthetic Oligonucleotide
aaagggcuucuuauucgggug

SEQ ID NO: 267 - RNA, 21 nt, Artificial Sequence
  Synthetic Oligonucleotide
uacuuuauacaaugucccuug

SEQ ID NO: 268 - RNA, 21 nt, Artificial Sequence
  Synthetic Oligonucleotide
aauguucuuccagaacccuua

SEQ ID NO: 269 - RNA, 21 nt, Artificial Sequence
  Synthetic Oligonucleotide
aauuauuaaagcccguacuua

SEQ ID NO: 270 - RNA, 21 nt, Artificial Sequence
  Synthetic Oligonucleotide
aauaaucugucgucuuugcug

SEQ ID NO: 271 - RNA, 21 nt, Artificial Sequence
  Synthetic Oligonucelotide
ucauuacacucaugaagccug

SEQ ID NO: 272 - RNA, 21 nt, Artificial Sequence
  Synthetic Oligonucelotide
uaaauaguggccugaaccuua

SEQ ID NO: 273 - RNA, 21 nt, Artificial Sequence
  Synthetic Oligonucleotide
cuaaauaguggccugaaccuu

SEQ ID NO: 274 - RNA, 21 nt, Artificial Sequence
  Synthetic Oligonucleotide
uuugcaguuugcaaucugcug

SEQ ID NO: 275 - RNA, 21 nt, Artificial Sequence
  Synthetic Oligonucleotide
ucaaaugugucauugagguug

SEQ ID NO: 276 - RNA, 21 nt, Artificial Sequence
  Synthetic Oligonucleotide
gcugauaaacuuaugggccuc

SEQ ID NO: 277 - RNA, 21 nt, Artificial Sequence
  Synthetic Oligonucleotide
guacuuuauacaaugucccuu

SEQ ID NO: 278 - RNA, 21 nt, Artificial Sequence
  Synthetic Oligonucleotide
cuuuauacaaugucccuuguc

SEQ ID NO: 279 - RNA, 21 nt, Artificial Sequence
  Synthetic Oligonucleotide
acuuaugaccacuucccaaua

SEQ ID NO: 280 - RNA, 21 nt, Artificial Sequence
  Synthetic Oligonucleotide
aaguuaucaugggacaccuuc

SEQ ID NO: 281 - RNA, 21 nt, Artificial Sequence
  Synthetic Oligonucleotide
auaaucgucagacacuuguuc

SEQ ID NO: 282 - RNA, 21 nt, Artificial Sequence
  Synthetic Oligonucleotide
auguucuguagggugacguug

SEQ ID NO: 283 - RNA, 21 nt, Artificial Sequence
  Synthetic Oligonucleotide
caguuuaaaugguuggcuguu

SEQ ID NO: 284 - RNA, 21 nt, Artificial Sequence
  Synthetic Oligonucleotide
ucaauuuggcuucuugacgug

SEQ ID NO: 285 - RNA, 21 nt, Artificial Sequence
  Synthetic Oligonucleotide
uauacaaugucccuugucauu

SEQ ID NO: 286 - RNA, 21 nt, Artificial Sequence
  Synthetic Oligonucleotide
cauaccaugugcuuccacuua

SEQ ID NO: 287 - RNA, 21 nt, Artificial Sequence
  Synthetic Oligonucleotide
ucgaugagcagauuugggauc

SEQ ID NO: 288 - RNA, 21 nt, Artificial Sequence
  Synthetic Oligonucleotide
acuucaacauguugacagguu

SEQ ID NO: 289 - RNA, 21 nt, Artificial Sequence
  Synthetic Oligonucleotide
uauuugugacagaacauguuu

SEQ ID NO: 290 - RNA, 21 nt, Artificial Sequence
  Synthetic Oligonucleotide
gauaaacuuaugggccucuua

SEQ ID NO: 291 - RNA, 21 nt, Artificial Sequence
  Synthetic Oligonucleotide
caaguuaucaugggacaccuu

SEQ ID NO: 292 - RNA, 21 nt, Artificial Sequence
  Synthetic Oligonucleotide
aguuuacacaaggcacagauu

SEQ ID NO: 293 - RNA, 21 nt, Artificial Sequence
  Synthetic Oligonucleotide
auaagguuggugagguuacuc

SEQ ID NO: 294 - RNA, 21 nt, Artificial Sequence
  Synthetic Oligonucleotide
cuauuugugacagaacauguu

SEQ ID NO: 295 - RNA, 21 nt, Artificial Sequence
  Synthetic Oligonucleotide
aacauuaucccaucagcuaug

SEQ ID NO: 296 - RNA, 21 nt, Artificial Sequence
  Synthetic Oligonucleotide
cucaaugcacuguuugcaguu

SEQ ID NO: 297 - RNA, 21 nt, Artificial Sequence
  Synthetic Oligonucleotide
caauggcauaccaugugcuuc

SEQ ID NO: 298 - RNA, 21 nt, Artificial Sequence
  Synthetic Oligonucleotide
cacauuuccagcuacuccaua

SEQ ID NO: 299 - RNA, 21 nt, Artificial Sequence
  Synthetic Oligonucleotide
gauuuguaagcaagaccaaug

SEQ ID NO: 300 - RNA, 21 nt, Artificial Sequence
  Synthetic Oligonucleotide
auugugaugaaaugaagguug

SEQ ID NO: 301 - RNA, 21 nt, Artificial Sequence
  Synthetic Oligonucleotide
uuacacagacuaacgacguug

SEQ ID NO: 302 - RNA, 21 nt, Artificial Sequence
  Synthetic Oligonucleotide
cacauccuguuaugcaccauc

SEQ ID NO: 303 - RNA, 21 nt, Artificial Sequence
  Synthetic Oligonucleotide
uuggguacuaucauccagcua

SEQ ID NO: 304 - RNA, 21 nt, Artificial Sequence
  Synthetic Oligonucleotide
ugaaucuagcaaauaguacuu

SEQ ID NO: 305 - RNA, 21 nt, Artificial Sequence
  Synthetic Oligonucleotide
cuauagagggacuucuacauc

SEQ ID NO: 306 - RNA, 21 nt, Artificial Sequence
  Synthetic Oligonucleotide
aguaagaaagccuguuuccuc

SEQ ID NO: 307 - RNA, 21 nt, Artificial Sequence
  Synthetic Oligonucleotide
ugcuuccacuuaugaccacuu

SEQ ID NO: 308 - RNA, 21 nt, Artificial Sequence
  Synthetic Oligonucleotide
ugauggugucauaugaagcug

SEQ ID NO: 309 - RNA, 21 nt, Artificial Sequence
  Synthetic Oligonucleotide
cauucuccuucagagaguguu

(19) United States
(12) Patent Application Publication
(10) Pub. No.: US 2013/0309238 A1
(43) Pub. Date: Nov. 21, 2013

(54) SIGNAL TRANSDUCTION PATHWAY MODULATION

(71) Applicants: Joerg Mattes, The Hill (AU); Paul Stephen Foster, Merewether (AU); Adam Collison, Maryland (AU); Luke Hatchwell, Adamstown (AU)

(72) Inventors: Joerg Mattes, The Hill (AU); Paul Stephen Foster, Merewether (AU); Adam Collison, Maryland (AU); Luke Hatchwell, Adamstown (AU)

(73) Assignee: NEWCASTLE INNOVATION LIMITED, Callaghan (AU)

(21) Appl. No.: 13/885,491

(22) PCT Filed: Nov. 15, 2011
(86) PCT No.: PCT/AU11/01474

§ 371 Date: Jul. 23, 2013

(30) Foreign Application Priority Data
Nov. 15, 2010 (AU) 2010905071

(51) Int. Cl. A61K 39/395; A61K 31/135; A61K 31/7088

(52) U.S. Cl. CPC A61K 39/3955; A61K 31/7088; A61K 31/135
USPC 424/139.1; 514/44 A; 514/646; 435/29

(57) ABSTRACT

Provided herein are methods and compositions for modulating signal transduction pathways by regulating the expression and/or activity of Midline-1, enabling the inhibition of airways inflammation, the inhibition of airways hyperresponsiveness, the inhibition of rhinovirus-associated inflammation, and reductions in cytokine and chemokine release. Methods and compositions disclosed herein facilitate the treatment and prevention of conditions associated with airway inflammation, airway tissue remodelling and rhinovirus-associated inflammation and symptoms, manifestations and exacerbations thereof, in particular of allergic diseases such as allergic airways diseases including asthma.

### FIELD OF THE INVENTION

The present disclosure relates generally to methods for modulating signal transduction pathways by regulating the expression and/or activity of Midline-1. The methods enable the inhibition of airways inflammation, the inhibition of airways hyperresponsiveness, the inhibition of rhinovirus-associated inflammation, and a reduction in cytokine and chemokine release thereby facilitating the treatment and prevention of conditions associated with airway inflammation, airway tissue remodelling and rhinovirus-associated inflammation and symptoms, manifestations and exacerbations thereof, in particular of allergic diseases such as allergic airways diseases including asthma.

### BACKGROUND OF THE DISCLOSURE

Asthma is one of the most widespread chronic health problems in the Western world and is increasing in prevalence around the world at an alarming rate. Australia has one of the highest rates of asthma in the world, with estimates suggesting that up to 10-20% of the population are affected. In addition to being potentially debilitating for sufferers, the direct and indirect costs of allergic airways diseases on health systems, families, businesses and economies are substantial.

Asthma is a chronic disease of the airways, however it is unknown whether inflammation initiates asthma or whether asthma initiates inflammation. In the lungs of healthy individuals inflammation is a common occurrence, and is in fact necessary to maintain normal lung homeostasis. In the lungs of an asthmatic, an exaggerated response to irritants occurs which results in an increased tendency to produce excessive airway narrowing (hyperresponsiveness). Increased airway inflammation follows exposure to inducers such as allergens, viruses, exercise, or non-specific irritant inhalation. Increased inflammation leads to exacerbations characterised by dyspnoea, wheezing, cough, and chest tightness. One aspect of asthma etiology that remains relatively poorly understood is the occurrence of airway tissue remodelling. Pathologically, airway remodelling appears to have a variety of features that include an increase in smooth muscle mass, mucus gland hyperplasia, persistence of chronic inflammatory cellular infiltrates, alterations in extracellular matrix deposition and release of fibrogenic growth factors.

Asthma is therefore a disease in which inflammation of the airways causes airflow obstruction and airway hyperresponsiveness, and in which structural changes or ‘remodelling’ of the surface of the airways takes place. Airway remodelling in diseases such as asthma is associated with hypertrophy and hyperplasia of cells such as airway smooth muscle cells and this can lead to a worsening of clinical symptoms.

Upon allergen sensing through Toll-like receptor 4 (TLR4) the respiratory epithelium and underlying mesenchyma release proinflammatory and growth factors into the airways that attract circulating blood cells. The production of innate proallergic cytokines such as thymic stromal lymphopoietin, granulocyte-macrophage colony—stimulating factor, IL-25, and IL-33 by airway epithelial cells promotes allergic lung inflammation and T cell maturation via activation of mucosal dendritic cells³. IL-13 production by T helper 2 (Th2) cells induces airways hyperresponsiveness or hypereactivity and mucus production in a STATE-dependent manner resulting in airway obstruction. Importantly activation of both TLR4 and STAT6 signaling pathways in the airway wall are essential for the development of salient features of allergic asthma (see, for example, Hammad et al., 2009 and Kuperman et al., 1998). Thus a bidirectional interaction between structural and immune cells is thought to underpin asthma expression and chronicity.

Presently asthma treatment is typically aimed at avoiding known allergens and respiratory irritants and controlling symptoms and airway inflammation through medication. This may include the use of short and/or long term treatment regimens. For short term ‘quick’ relief, for example during an asthma attack, short-acting bronchodilators may be employed. For longer term control measures, medications include, for example, inhaled steroids, typically corticosteroids to prevent inflammation, leukotriene inhibitors, long-acting bronchodilators to help keep airways open, a combination of corticosteroid and bronchodilator, using either separate inhalers or a single inhaler (e.g. fluticasone/salmeterol and budesonide/formoterol), and antibodies to neutralise immunoglobulin E (IgE) or interleukin-5 (IL-5) (e.g. omalizumab). Alternative current therapies aim to inhibit the constriction of airways by stimulating beta-2 receptors in the airways via short or long-acting beta-2 receptor agonists (“relievers” and “controllers” respectively).

However, existing therapies do not address the complex nature of the pathways that are activated in the airways during asthma on a molecular level. Rather they aim to either suppress only one or a few out of numerous disease mechanisms that promote aberrant immune responses or alleviate symptoms. Furthermore current therapies are commonly associated with significant side effects (for example in the case of steroid use) or tachyphylaxis (for example following administration of long-acting beta-2 receptor antagonists).

There is a clear need not only for effective therapies for the treatment and management of allergic airways diseases such as asthma, but also for strategies and approaches to prevent the onset and development of such diseases and to control and minimise the symptoms and exacerbations associated with allergic airways diseases such as asthma.

### SUMMARY OF THE DISCLOSURE

The present disclosure is predicated on the inventors' surprising findings described herein that Midline-1 expression is upregulated relative to normal endogenous levels in airway tissues, in particular in airway epithelial cells, upon allergen exposure and that this increase in expression is promoted by TLR4/MyD88-dependent allergen sensing and the resultant activation of TRAIL. Moreover, inhibition of Midline-1 using a Midline-1 antagonist or a PP2A agonist in a mouse model of allergic airways disease abolished airways hyperresponsiveness, suppressed airways inflammation and mucous production, and reduced Th2 cytokine release.

These findings enable the development of methods for treating and preventing allergic conditions, including allergic airways diseases such as asthma, and methods of reducing the incidence and/or severity of manifestations associated with allergic diseases such as asthma, including airway tissue remodelling, airway hyperresponsiveness, airway inflammation, mucous production and cytokine/chemokine release. In the context of asthma these findings are valuable in that they provide a means of reducing the severity of some of the more serious consequences of airway diseases such as asthma and of preventing the onset of asthma exacerbations. The present inventors' findings also provide novel means for diagnosing the occurrence of, or predisposition to, allergic airway diseases such as asthma, based for example on an analysis of Midline-1 mRNA or protein expression levels.

According to one aspect of the disclosure there is provided a method for treating or preventing a condition associated with airway inflammation and/or airway tissue remodelling, or at least one symptom, manifestation or exacerbation of the condition, the method comprising administering to a subject in need thereof an effective amount of an agent capable of inhibiting the expression and/or activity of Midline-1.

According to a further aspect of the disclosure there is provided a method for treating or preventing an allergic condition or at least one symptom, manifestation or exacerbation of the condition, the method comprising administering to a subject in need thereof an effective amount of an agent capable of inhibiting the expression and/or activity of Midline-1.

In a particular embodiment, the condition is an allergic airways disease. The subject may suffer from, or be predisposed to, the condition. If suffering from the condition, the subject may be symptomatic or asymptomatic. The allergic airways disease may be, for example, asthma, including asthma exacerbations, chronic obstructive pulmonary disease (COPD), pulmonary fibrosis, cystic fibrosis, and a wheezing illness. The asthma exacerbations may be rhinovirus-associated exacerbations.

In accordance with embodiments of the present disclosure, the administration of the agent may, for example, inhibit, reduce or prevent the establishment of airways hyperresponsiveness, suppress or inhibit airways inflammation or mucous production, reduce the release of cytokines such as Th2 or chemokines such as CCL20, inhibit collagen deposition in airway cells and/or inhibit or prevent airways fibrosis.

The inhibition of the expression and/or activity of Midline-1 may occur in one or more cells, typically abnormal cells implicated in the condition. The cells may be airway epithelial cells.

The agent may inhibit the expression and/or activity of Midline-1 directly or indirectly. In inhibiting expression or activity directly, the agent may interact with the Midline-1 at the pre-transcriptional level, post-transcriptional level or post-translational level.

In one embodiment, the agent may be a molecule(s) capable of inhibiting or suppressing expression of Midline-1. Inhibition of expression may be at the level of the nucleotide sequence encoding Midline-1 and the inhibitor may be an antisense construct such as a small interfering RNA (siRNA), catalytic antisense construct, morpholino or other antisense oligonucleotide. The Midline-1 may comprise the amino acid sequence as set forth in SEQ ID NO:1. The Midline-1 may be encoded by the nucleotide sequence as set forth in SEQ ID NO:2. In an exemplary embodiment the inhibitor is an siRNA molecule comprising a sequence as set forth in SEQ ID NO:3.

In an alternate embodiment, the agent may be an inhibitor or antagonist of the activity of the Midline-1 polypeptide. The antagonist may be an antibody, such as a monoclonal antibody.

In an alternate embodiment, the agent may inhibit Midline-1 expression or activity indirectly by exerting its effect on a molecule or molecules upstream of Midline-1 in a signal transduction or other biochemical pathway, which molecule or molecules thereby act, directly or indirectly, to inhibit the expression or activity of Midline-1. In one exemplary embodiment, the agent exerting an indirect effect on Midline-1 may interact with PP2A. By way of example, the agent may be a compound capable of activating PP2A, optionally selectively activating PP2A. By selectively activating PP2A the compound may bind PP2A but not sphingosine-1-phosphate receptors, such as is the case with the compound 2-amino-4-(4-heptyloyphenol)-2-methylbutanol (AAL_{(S)}).

Inhibiting Midline-1 expression or activity, whether achieved directly or indirectly, will typically comprise modulating the Midline-1 in airway tissue of the subject to normalising Midline-1 levels in said tissue relative to normal endogenous levels.

Provided herein are methods for the treatment or prevention of asthma, and the treatment or prevention of one or more asthma exacerbations or symptoms of the disease.

According to another aspect of the disclosure there is provided the use of an agent capable of inhibiting the expression and/or activity of Midline-1 in the manufacture of a medicament for the treatment or prevention of a condition associated with airway inflammation and/or airway tissue remodelling, or at least one symptom, manifestation or exacerbation of the condition.

Also provided is the use of an agent capable of inhibiting the expression and/or activity of Midline-1 in a method for treating or preventing a condition associated with airway inflammation and/or airway tissue remodelling, or at least one symptom, manifestation or exacerbation of the condition.

Also provided herein are pharmaceutical compositions for use in the treatment or prevention of a condition associated with airway inflammation and/or airway tissue remodelling, or at least one symptom, manifestation or exacerbation of the condition, the composition comprising an agent capable of inhibiting the expression and/or activity of Midline-1, optionally together with one or more pharmaceutically acceptable carriers, diluents or excipients.

According to another aspect the present disclosure provides a method for diagnosing a condition associated with airway inflammation and/or airway tissue remodelling, or susceptibility or predisposition thereto, in a subject, the method comprising determining the level of Midline-1 in a fluid or in airway tissue or cells of the subject.

The fluid may be serum or bronchoalveolar lavage fluid. The cells may be airway epithelial cells.

The method may comprise isolating fluid or airway cells from the subject and determining the level of expression of Midline-1 in the fluid or airway cells, wherein the level of expression of the Midline-1 is indicative of the condition, or a susceptibility or predisposition thereto.

According to another aspect of the disclosure there is provided a method for screening for substances which modulate Midline-1, the method comprising:
(a) contacting at least one candidate substance with a cell expressing Midline-1;
(b) determining whether the candidate substance modulates Midline-1 expression and/or activity; and
(c) selecting the substance which modulates Midline-1 expression and/or activity.

As disclosed and exemplified herein, the present inventors have also found that inhibition of Midline-1 protects allergic and non-allergic mice from rhinovirus-associated airway hyperresponsiveness (AHR) and inflammation. Accordingly, a further aspect of the present disclosure provides a method for treating or preventing rhinovirus-associated inflammation, the method comprising administering to a subject in need thereof an effective amount of an agent capable of inhibiting the expression and/or activity of Midline-1. The rhinovirus-associated inflammation may be allergic inflammation. However also in accordance with this aspect the rhinovirus-associated inflammation may be independent of any existing allergy.

A further aspect of the present disclosure provides a method for inhibiting or preventing fibrosis, the method comprising administering to a subject in need thereof an effective amount of an agent capable of inhibiting the expression and/or activity of Midline-1.

### BRIEF DESCRIPTION OF THE DRAWINGS

Embodiments of the disclosure are described herein, by way of non-limiting example only, with reference to the following drawings.

FIG. 1: (a) Total lung resistance as percentage change of baseline measurement (water) in response to inhaled methacholine in allergic (HDM) versus non-allergic (SAL) mice. Results are mean±s.e.m. (n=6-10 mice per group). (b) Number of cells in bronchoalveolar lavage fluid (BALF). Results are mean±s.e.m. (n=3-4 mice per group) (c) Number of peribronchial perivacular eosinophils (×1000) and mucus-producing cells (×400) per high-power field (HPF). Results are mean±s.e.m. (n=3 mice per group). (d) Cytokine release from in-vitro house dust mite stimulated peribronchial lymphnode cells and (e) CCL20 levels in lung homogenates. Results are mean s.e.m. (n=4 mice per group). (f) Midline-1 mRNA and (g) protein expression in the airway wall. Scale Bar, 25 μm. (h) PP2A activity and (i) PP2Ac levels in lung homogenates. (n=3-4 mice per group). *, P<0.05 and **, P<0.01.

FIG. 2: (a) Midline-1 mRNA and (b) protein expression in the airway wall of non-allergic (SAL) versus allergic (HDM) mice treated with a scrambled siRNA (Nonsense siRNA) or a Midline-1 targeting siRNA (MID-1 siRNA) every second day during the allergen challenge period intranasally. Results are mean±s.e.m. (n=3-4 mice per group). (c) Total lung resistance as percentage change of baseline measurement (water) in response to inhaled methacholine. Results are mean±s.e.m. (n=6-10 mice per group). (d) Number of peribronchial perivacular eosinophils (×1000) and mucus-producing cells (×400) per high-power field (HPF). Results are mean±s.e.m. (n=3 mice per group). (e) Cytokine release from in-vitro house dust mite stimulated peribronchial lymphnode cells and (f) CCL20 expression in airway wall. Results are mean±s.e.m. (n=4-6 mice per group). (g) PP2A activity and (h) PP2Ac levels in lung homogenates. (n=3 mice per group). (i) Phosphorylated p38 MAPK protein expression in the airway wall. Scale Bar, 25 μm. *, P<0.05 and **, P<0.01.

FIG. 3: (a) PP2A activity in lung homogenates from non-allergic (SAL) versus allergic (HDM) mice treated with 2% (2-hydroxypropyl)—cyclodextrin (vehicle) or AAL(S) each day during the allergen challenge period intranasally. Results are mean±s.e.m. (n=4 mice per group). (b) Total lung resistance as percentage change of baseline measurement (water) in response to inhaled methacholine. Results are means.e.m. (n=5-8 mice per group). (c) Number of cells in bronchoalveolar lavage fluid (BALF). Results are mean±s.e.m. (n=3-4 mice per group). (d) Cytokine release from in-vitro house dust mite stimulated peribronchial lymphnode cells and (e) CCL20 levels in lung homogenates. Results are means.e.m. (n=4 mice per group). Scale Bar, 25 μm. *, P<0.05 and **, P<0.01.

FIG. 4: Midline-1 inhibition ameliorates rhinovirus-associated airways inflammation, obstruction, and asthma exacerbation. (a) Total lung resistance as percentage change of baseline measurement (water) in response to inhaled methacholine. Results are mean±s.e.m. (n=6-10 mice per group). (b) Number of peribronchial perivacular eosinophils (×1000) and mucus-producing cells (×400) per high-power field (HPF). Results are mean±s.e.m. (n=2-3 mice per group). (c) Midline-1 and CCL20 mRNA, (d) Positive strand RV1B RNA, and (e) IFN mRNA in the airway wall of naïve mice treated with a scrambled siRNA (Nonsense siRNA) or a Midline-1 targeting siRNA (MID-1 siRNA) 24 hrs before RV1B challenge intranasally. Results are mean s.e.m. (n=3-4 mice per group). *, P<0.05 and **, P<0.01.

FIG. 5: Midline-1 and PP2A in house dust mite, TRAIL, and rhinovirus exposed human airway epithelial cells. (a) TRAIL, Midline-1, CCL20 expression and (b) PP2A activity in BEAS-B2 cells stimulated with house dust mite extract or recombinant TRAIL (1000 ng/ml). Results are mean±s.e.m. and representative for n=3 experiments. (c) Immunoprecipitation for PP2Ac in unstimulated BEAS-B2 cell lysates. Total lysate (lane one), PP2Ac precipitant (lane two), PP2Ac-depleted lysate (lane three). (d) TRAIL, Midline-1, and CCL20 expression in primary airway epithelial cells after RV 1B or UV-RV1B infection. Results are mean±s.e.m. (n=5 asthmatics and n=5 non-asthmatic controls). *, P<0.05. (e) Correlation between TRAIL and Midline-1 expression in RV1B infected airway epithelial cells. Results are mean±s.e.m. *, P<0.05.

FIG. 6: PP2A activity in immortalised human bronchial epithelial cells stimulated with crude house dust mite extract (HDM) and treated with salbutamol, mitoxantrone analogue or AAL₄), compared to HDM and saline (HDM+Veh). Data collated from three independent experiments. *, P<0.05 and **, P<0.01.

FIG. 7: BALF levels, AHR and CCL20 levels in non-allergic and HDM-allergic and challenged mice treated with FTY720, pFTY720 or saline (SAL). *, P<0.05; **, P<0.01; ***, P<0.005.

FIG. 8: (a) Collagen and smooth muscle alpha actin deposition surrounding the airways and (b) lung function (FVC, forced vital capacity; FEV, forced expiratory volume) in wildtype (WT) and TRAIL −/− (T−/−) mice challenged with ovalbumin (OVA) and treated with AAL_{(S)} or saline (SAL). (c) Collagen deposition surrounding the airways in wildtype (WT) and TRAIL −/− (T−/−) mice administered bleomycin (Bleo) intratracheally and treated with AAL_{(S)} or saline (SAL). *, P<0.05 and **, P<0.01.

The subject specification contains amino acid and nucleotide sequence information prepared using the programme PatentIn Version 3.4, presented herein in a Sequence Listing. Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. The amino acid sequence of human Midline-1 is provided in SEQ ID NO:1 and the nucleotide sequence of the gene encoding human Midline-1 is provided in SEQ ID NO:2. SEQ ID NO:3 provides the sequence of an exemplary anti-Midline-1 siRNA molecule for use in accordance with the present disclosure. SEQ ID NO's: 4 to 11 provide the oligonucleotide primer sequences exemplified herein. SEQ ID NO's: 12 to 309 provide the sequences of predicted anti-Midline-1 siRNA molecules for use in accordance with the present disclosure.

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

In the context of this specification, the term “activity” as it pertains to a protein, polypeptide or polynucleotide means any cellular function, action, effect or influence exerted by the protein, polypeptide or polynucleotide, either by a nucleic acid sequence or fragment thereof, or by the protein or polypeptide itself or any fragment thereof. The cellular function, action, effect or influence may be effected by the protein, polypeptide or polynucleotide may be exerted directly or indirectly.

By “airway tissue” is meant the tissue of the passages which run from the back of the mouth and nose into the lungs, together with the alveoli. The largest of these passages is the trachea (also known as the “windpipe”). In the chest, the trachea divides into two smaller passages termed the bronchi, each of these being further characterised by three regions termed the primary bronchus, secondary bronchus and tertiary bronchus. Each bronchus enters one lung and divides further into narrower passages termed the bronchioles. The terminal bronchiole supplies the alveoli. This network of passages are often colloquially termed the “bronchial tree” and, in the context of asthma, undergo inflammation, muscle constriction and swelling of their lining leading to a reduction in airflow into and out of the lungs. It is this tissue which also ultimately undergoes remodelling, thereby leading to still further complications in terms of the irreversible reduction of lung functioning.

As used herein the term “associated with” when used in the context of a disease or condition “associated with” airway inflammation or airway tissue remodelling means that the disease or condition may result from, result in, be characterised by, or otherwise associated with airway inflammation or airway tissue remodelling. Thus, the association between the disease or condition and airway inflammation or airway tissue remodelling may be direct or indirect and may be temporally and/or spatially separated. Those skilled in the art will appreciate that reference to a condition “associated with” airway inflammation or airway tissue remodelling does not necessarily imply that any individual to be treated or diagnosed in accordance with the present disclosure will display airway inflammation or airway tissue remodelling, but rather that these features are typically or generally associated with a manifestation of the condition in most or many sufferers.

As used herein the term “rhinovirus-associated inflammation” means inflammation that results from, is induced by, or is otherwise associated with a rhinovirus infection in a subject. The inflammation may be of the airways, including the upper and lower respiratory tract, or may be of another organ or tissue.

As used herein, the term “disease control” means the status of the disease or disorder, typically in light of intervention to treat the disease or disorder. Thus “disease control” describes the range and severity of symptoms and conditions experienced and suffered by patients as a result of their disorder. Disease control effectively provides a measure at a given point in time of the disease status of an individual, reflecting both current therapeutic treatment regimes used by the individual and the individual's recent experiences.

As used herein the term “effective amount” includes within its meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

It will be understood that as used herein the term “expression” may refer to expression of a polypeptide or protein, or to expression of a polynucleotide or gene, depending on the context. The polynucleotide may be coding or non-coding. Expression of a polynucleotide may be determined, for example, by measuring the production of RNA transcript levels. Expression of a protein or polypeptide may be determined, for example, by immunoassay using an antibody(ies) that bind with the polypeptide.

The term “inhibiting” and variations thereof such as “inhibition” and “inhibits” as used herein do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably.

In the context of the present disclosure, the term “inhibitor” as used in relation to Midline-1 refers to any agent or action capable of inhibiting either or both the expression and activity of Midline-1, either directly or indirectly. Accordingly the inhibitor may operate directly or indirectly on the Midline-1 polypeptide, the corresponding mRNA or gene, or alternatively act via the direct or indirect inhibition of any one or more components of a Midline-1-associated pathway. Such components may be molecules activated, inhibited or otherwise modulated prior to, in conjunction with, or as a consequence of Midline-1 activity. Thus, the inhibitor may operate to prevent transcription, translation, post-transcriptional or post-translational processing or otherwise inhibit the activity of Midline-1 or a component of a Midline-1-associated pathway in any way, via either direct or indirect action. The inhibitor may for example be nucleic acid, peptide, any other suitable chemical compound or molecule or any combination of these. It will be understood that in indirectly impairing the activity of Midline-1 or a component of a Midline-1-associated pathway, the inhibitor may effect the activity of molecules which regulate, or are themselves subject to regulation or modulation by, Midline-1 or a component of a Midline-1-associated pathway. Thus encompassed by the term Midline-1 inhibitors are, for example, agonists of the protein phosphatase 2, PP2A.

As used herein the term “Midline-1” refers to the human protein or polypeptide commonly referred to as Midline-1 having E3 ubiquitin ligase activity and targeting the catalytic subunit of protein phosphatase 2 for degradation, and to homologues, orthologues, derivatives, variants and functional fragments thereof that share substantially the same or similar activity. Thus, whilst typically referring to the polypeptide, and the encoding gene, as found in humans, or to derivatives, fragments or variants thereof, those skilled in the art will appreciate that homologues of human Midline-1 from other species are also contemplated and encompassed by the present disclosure. Midline-1 may also be referred to in the art as Tripartite motif-containing protein 18, Putative transcription factor XPRF, Midin, RING finger protein 59 and Midline 1 RING finger protein. The gene encoding human Midline-1 is also known as MID1 and may be referred to in the art as FXY, RNF59, TRIM18 and XPRF. The Midline-1 polypeptide and the encoding gene, by all of the various alternative names and designations, are contemplated and encompassed by the present disclosure. The amino acid sequence of human Midline-1 is located in the UniProtKB/Swiss-Prot database under accession number 015344, and is provided in the present disclosure in SEQ ID NO:1. The cDNA encoding human Midline-1 is located in the EMBL database under accession number Y13667, and is provided in the present disclosure in SEQ ID NO:2. The term Midline-1 may be used herein to refer to either or both the Midline-1 polypeptide or the gene encoding the Midline-1 polypeptide, interchangeably. Those skilled in the art will recognize from the context of the disclosure whether the polypeptide or polynucleotide (gene) is the subject of the discussion.

Reference to “normal endogenous levels” should be understood as a reference to the level of Midline-1 which is expressed in the airway tissue of a subject who is not suffering from nor is predisposed to a condition associated with aberrant airway inflammation and/or airway tissue remodelling. It would be appreciated by the person of skill in the art that this “normal level” is likely to correspond to a range of levels, as opposed to a singularly uniform discrete level, due to differences between cohorts of individuals. By “cohort” is meant a cohort characterised by one or more features which are also characteristic of the subject who is undergoing treatment. These features include, but are not limited to, age, gender or ethnicity, for example. Accordingly, reference herein to modulating Midline-1 levels relative to normal endogenous levels is a reference to increasing or decreasing airway tissue Midline-1 levels relative to either a discrete Midline-1 level which may have been determined for normal individuals who are representative of the same cohort as the individual being treated or relative to a defined Midline-1 level range which corresponds to that expressed by a population of individuals corresponding to those from a range of different cohorts.

As used herein the term “polypeptide” means a polymer made up of amino acids linked together by peptide bonds. The terms “polypeptide” and “protein” are used interchangeably herein, although for the purposes of the present disclosure a “polypeptide” may constitute a portion of a full length protein. The term “polynucleotide” as used herein refers to a single- or double-stranded polymer of deoxyribonucleotide, ribonucleotide bases or known analogues or natural nucleotides, or mixtures thereof. In some contexts in the present specification the terms “polynucleotide” and “nucleic acid molecule” are used interchangeably.

The term “subject” as used herein refers to mammals and includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human.

Reference to “susceptibility” should be understood as a reference to both determining whether any existing symptoms associated with or indicative of a condition associated with airways inflammation or airway tissue remodeling, or of an allergic airways disease, experienced by an individual are linked to abnormal Midline-1 levels as described herein and to determining whether individuals who have not experienced symptoms indicative of such a disorder nevertheless exhibit a predisposition or risk thereto. Thus, depending on the particular circumstances of a particular subject, the term “susceptibility” should be understood to mean vulnerability to a condition associated with airways inflammation or airway tissue remodeling, or having an increased likelihood of development of a condition associated with airways inflammation or airway tissue remodeling in the future.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of some disorders, methods of the present disclosure involve “treating” the disorder in terms of reducing or ameliorating the occurrence of a highly undesirable event associated with the disorder or an irreversible outcome of the progression of the disorder but may not of itself prevent the initial occurrence of the event or outcome. Accordingly, treatment includes amelioration of the symptoms of a particular disorder or preventing or otherwise reducing the risk of developing a particular disorder.

The present disclosure provides methods for inhibiting airways inflammation, inhibiting airways hyperresponsiveness and reducing cytokine and chemokine release in airway cells and tissues, thereby facilitating the treatment and prevention of conditions associated with airway inflammation and airway tissue remodelling and symptoms, manifestations and exacerbations thereof, in particular of allergic airways diseases such as asthma.

The present disclosure also provides methods for preventing and treating allergic conditions, in particular allergic conditions associated with inflammation. Also provided are methods for preventing and treating rhinovirus-associated inflammation and rhinovirus-associated exacerbations of allergic diseases such as asthma.

Without wishing to be bound by theory, the inventors propose that TRAIL-induced Midline-1 links TLR4-dependent sensing of allergens by airway epithelial cells to hallmark features of allergic asthma, including airway obstruction and allergic inflammation by modulating PP2A activity. Targeting Midline-1 and/or increasing PP2A activity in the airway wall of subjects with asthma or wheezing illness may therefore be of potential therapeutic benefit.

The findings described herein also offer novel diagnostic target and approach for the diagnosis of conditions associated with aberrant airway tissue remodelling such as asthma, and for the identification of predisposition to such conditions. Tissue remodelling is the unwanted and undesirable outcome of some airway diseases such as asthma, chronic obstructive pulmonary disease (COPD), pulmonary fibrosis including idiopathic pulmonary fibrosis, and cystic fibrosis. Accordingly, in the absence of the development of cures for these diseases, the development by the present inventors of means to reduce one or more of the symptoms, manifestations or exacerbations associated with and characteristic of such diseases is a crucial finding enabling the rational design of therapeutic and prophylactic methods for reducing the occurrence or severity of many airway diseases.

Accordingly, one aspect of the present disclosure provides a method for treating or preventing a condition associated with airway inflammation and/or airway tissue remodelling, or at least one symptom, manifestation or exacerbation of the condition, the method comprising administering to a subject in need thereof an effective amount of an agent capable of inhibiting the expression and/or activity of Midline-1

A further aspect of the disclosure provides a method for treating or preventing an allergic condition or at least one symptom, manifestation or exacerbation of the condition, the method comprising administering to a subject in need thereof an effective amount of an agent capable of inhibiting the expression and/or activity of Midline-1.

A further aspect provides a method for treating or preventing rhinovirus-associated inflammation, the method comprising administering to a subject in need thereof an effective amount of an agent capable of inhibiting the expression and/or activity of Midline-1.

A further aspect of the present disclosure provides a method for inhibiting or preventing fibrosis, the method comprising administering to a subject in need thereof an effective amount of an agent capable of inhibiting the expression and/or activity of Midline-1.

Other aspects of the disclosure provide methods for the diagnosis of conditions associated with airway inflammation and/or airway tissue remodelling, or susceptibility or predisposition thereto, comprising determining the level of Midline-1 in a fluid or airway tissue or cells of a subject.

Embodiments of the disclosure also provide pharmaceutical compositions and diagnostic kits for use in accordance with the methods disclosed herein.

As detailed herein, embodiments of the present disclosure are applicable to the treatment, prevention and/or diagnosis of conditions associated with aberrant airway inflammation and/or airway tissue remodelling. Such conditions include, but are not limited to, asthma, chronic obstructive pulmonary disease (COPD), pulmonary fibrosis, cystic fibrosis and wheezing illnesses. Those skilled in the art will appreciate that any condition, disorder or disease that is associated with unwanted inflammation, remodelling or hyperresponsiveness of the airways, for example to inhaled allergens, is contemplated by the present disclosure and may be treated, prevented or diagnosed in accordance with embodiments disclosed herein.

In terms of modulating Midline-1 levels, embodiments disclosed herein provide for the downregulation or reduction of airway tissue Midline-1 levels in order to approach the normal endogenous levels. To this end, however, it should be understood that the Midline-1 level need not necessarily be fully normalised in order to achieve the desired outcome, although complete normalisation is typically desirable. Merely partially decreasing Midline-1 levels may at least ameliorate the incidence or severity of a condition in the subject. It should also be understood that the methods of the present disclosure may be applied transiently or in an ongoing manner depending on the requirements of the particular situation. Further, it will be appreciated that there may be circumstances in which it is desirable or beneficial to reduce levels of Midline-1 beyond normal endogenous levels. Such reduction of Midline-1 levels is contemplated and encompassed by the present application.

As would be appreciated by those skilled in the art in some circumstances it may be desirable to induce or upregulate the occurrence of airway inflammation and/or tissue remodelling, for example in an in vitro model or an animal model, in order to facilitate an outcome such as providing a system for screening for the effectiveness of adjunctive therapies, prophylactic therapies or for otherwise facilitating the ongoing research into allergic airways diseases. To this end, one may achieve this outcome by increasing the endogenous Midline-1 levels of the subject airway tissue. Reference to “increasing” in this regard should be understood to have an analogous meaning to “normalising” in that said increase may be partial or total and will depend on the extent to which one is seeking to facilitate the occurrence of the desired event.

Those skilled in the art will appreciate that methods of the present disclosure may be performed in vivo, ex vivo or in vitro. Although methods are typically to therapeutically or prophylactically treat an individual in vivo, it should nevertheless be understood that it may be desirable that a method of the disclosure be applied in an ex vivo or in vitro environment, such as in the contexts detailed above.

The Midline-1 will typically, in accordance with the present disclosure, be human Midline-1 and may comprise an amino acid sequence as set forth in SEQ ID NO:1, or be encoded by a polynucleotide comprising a nucleotide sequence as set forth in SEQ ID NO:2. The present disclosure also contemplates agents that are capable of inhibiting the expression and/or activity of variants and functional fragments of Midline-1. A variant of Midline-1 typically refers to a molecule which exhibits at least some of the functional activity of the Midline-1 of which it is a variant. A variant may take any form and may be naturally or non-naturally occurring. Variants include homologues, meaning that the molecule is derived from a species other than human.

Embodiments disclosed herein contemplate the administration of one or more agents capable of inhibiting or reducing the expression and/or activity of Midline-1. Such inhibitors may directly or indirectly effect Midline-1 expression and may act at the level of the gene encoding Midline-1 or any product thereof including Midline-1 mRNA or Midline-1 polypeptide. The inhibitor may be a proteinaceous or non-proteinaceous molecule that modulates the transcription and/or translation of the gene encoding Midline-1 or a functional portion thereof (such as a promoter region), or alternatively that modulates the transcription and/or translation of an alternative gene or functional portion thereof, which alternative gene or gene product directly or indirectly modulates the expression of Midline-1. The inhibitory agent may be an antagonist. Antagonists may be any compound capable of blocking, inhibiting or otherwise preventing Midline-1 from carrying out its normal biological function. For the present purposes, the term “antagonist” is used hereinafter to refer to inhibitors of Midline-1 activity and Midline-1 expression.

A variety of suitable antagonists may be employed and the scope of the disclosure is not limited by the selection of any one particular molecule or compound. Suitable antagonists include antibodies, such as monoclonal antibodies, and antisense nucleic acids which prevent transcription or translation of Midline-1 genes or mRNA. Modulation of expression may also be achieved utilising antigens, RNA, ribozymes, DNAzymes, aptamers, antibodies or molecules suitable for use in cosuppression.

An antibody in the context of a Midline-1 antagonist, refers to an immunoglobulin or a fragment or a derivative thereof and encompasses any polypeptide comprising an antigen-binding site. In particular, the antigen may be an antigen of Midline-1 or an antigen from a protein involved in the transcription or translation of Midline-1 genes or mRNA. The antibody may be produced in vitro or in vivo. The antibody may be polyclonal, monoclonal, monospecific, polyspecific, non-specific, humanized, single-chain, chimeric, synthetic, recombinant, hybrid, mutated, or grafted. Antibodies are generally tetrameric glycosylated proteins comprising two light (L) chains (approximately 25 kDa each) and two heavy (H) chains (approximately 50-70 kDa each). A variable domain largely responsible for antigen binding is typically present in each of the H and L chains. The H and L chains further comprise constant domains primarily responsible for effector function. There are two types of human L chains, classified as kappa and lambda. H chains are classified as mu, delta, gamma, alpha, or epsilon based upon the constant domain amino acid sequence, defining the antibody's isotype as IgM, IgD, IgG, IgA, and IgE, respectively.

The antibody may be an antibody fragment such as a Fab, F(ab′)₂, Fv, scFv, Fd, dAb, and other antibody fragments which retain an antigen-binding function.

Antigen-binding domains typically comprise an antibody light chain variable domain (VL) and an antibody heavy chain variable domain (VH). In some embodiments an antigen-binding domain may comprise only a VL region or a VH domain. For example, an Fd may comprise only of a VH domain but retains an antigen-binding function.

Fab fragments contain the antigen binding portion of a complete antibody and may consist of the L chain disulfide bonded to a portion of the H chain comprising the V domain and first constant domain. Single chain Fv antibody fragments (scFv) comprise the VH variable domain is linked to the VL domain by a polypeptide linker. Antibody fragments such as Fab and scFv molecules having sequences derived from germline or affinity matured V domains of antibody binding specifically to an epitope.

The antibodies, fragments or derivatives thereof may bind an epitope with an affinity (Kd)) from about 10⁻⁶ M to about 10⁻¹⁴ M . In some embodiments the antibody or fragments thereof may bind an epitope with an affinity of about 10⁻⁷M or greater. In another embodiment the antibody or fragments thereof may bind an epitope with an affinity of about with a Kd less than about 10⁻⁷, 10⁻⁸, 10⁻⁹, 10⁻¹⁰, 10⁻¹¹, 10⁻¹², 10¹³ or 10⁻¹⁴M.

The antibodies, fragments or derivatives thereof may be linked to another molecule, such as a protein or peptide (e.g. albumin), anti-inflammatory agent or immunomodulatory agent. The antibodies, fragments or derivatives thereof may be linked by chemical cross-linking or by recombinant methods. The polymer may be, for instance polyethylene glycol, polypropylene glycol, or polyoxyalkylenes. The antibodies or fragments thereof may be chemically modified by covalent conjugation to a polymer, for example, to increase their circulating half-life.

Small molecule and other naturally occurring and synthetically derived chemical agonists of PP2A are also contemplated for use in accordance with the present disclosure, for example as a means of indirectly modulating Midline-1. In an embodiment the small molecule may be selected from FTY720 and AAL_{(S)}. In a particular embodiment the small molecule agonist may be a selective activator of PP2A, for example an activator that interacts with PP2A but not sphingosine-1-phosphate receptors. In a particular embodiment the selective activator of PP2A is AAL_{(S)}. Those skilled in the art will appreciate that a variety of other agonists of PP2A may also be used in accordance with the present disclosure, and the scope of the present disclosure is not limited by reference to any specific agonists. Suitable exemplary PP2A agonists include beta-adrenergic receptor agonists (including salbutamol), mitoxantrone, sodium selenate, sodium selenite, forskolin, sorafenib (optionally in combination with bortezomib), dithiolethione, curcumin, ceramide, aldosterone, and agents to induce hyperosmotic stress (such as dextran sulphate sodium).

Suitable antisense constructs for use in accordance with the present disclosure include antisense oligonucleotides, small interfering RNAs (siRNAs) and catalytic antisense nucleic acid constructs, the production and use of which are well known to those skilled in the art. One particularly suitable antisense technology, known as RNA interference (RNAi), see, eg. Chuang et al. (2000) PNAS USA 97: 4985) may be used, according to known methods in the art (for example Hammond et al. (2000) Nature 404: 293-296; Bernstein et al. (2001) Nature 409: 363-366; Elbashir et al (2001) Nature 411: 494-498; WO 99/49029 and WO 01/70949, the disclosures of which are incorporated herein by reference), to inhibit the expression or activity of nucleic acid molecules encoding Midline-1. RNAi refers to a means of selective post-transcriptional gene silencing by destruction of specific RNA by small interfering RNA molecules (siRNA). The siRNA is generated by cleavage of double stranded RNA, where one strand is identical to the message to be inactivated. Double-stranded RNA molecules may be synthesised in which one strand is identical to a specific region of the Midline-1 transcript and introduced directly. By way of example only, a suitable anti-Midline-1 siRNA molecule suitable for use in accordance with embodiments of the disclosure to reduce Midline-1 levels comprises a sequence as set forth in SEQ ID NO:3. Other siRNA molecules suitable for use in accordance with embodiments of the disclosure to reduce Midline-1 levels may be designed by any method known in the art. For example, suitable siRNA may be designed in accordance with the so-called Tuschl's Rules (see Tuschl, T. in RNAi, a guide to gene silencing, Hannon, G. J. (ed.), Cold Spring Harbor Laborotory Press, Cold Spring Harbor, N.Y., 265-295 (2003)) and principles of rational siRNA design such as those set down in Reynolds, A. et al., (2004) Nature Biotechnol. 22: 326-330. A variety of algorithms and web-based tools are also available and well known to those skilled in the art for designing siRNAs. Examples include the application provided by the Whitehead Institute based on methods described in Bingbing et al (2004) Nucleic. Acids. Res. 32: W130-W134. Exemplary siRNA sequences include the antisense sequences as set forth in SEQ ID NO's: 12 to 309.

RNAi agents may be siRNAs (synthetic RNAs) or DNA-directed RNAs (ddRNAs). siRNAs may be manufactured by methods known in the art including oligonucleotide synthesis. In some embodiments synthesized RNAi agents incorporate chemical modifications to increase half life and/or efficacy of the siRNA agent and/or allow a more robust delivery formulation. The modifications include incorporation of a polycyclic sugar surrogate; such as a cyclobutyl nucleoside, cyclopentyl nucleoside, proline nucleoside, cyclohexene nucleoside, hexose nucleoside or a cyclohexane nucleoside; inclusion of a non-phosphorous-containing internucleoside linkage; modification of a 2′ substituent group on a sugar moiety that is not H or OH; a modified base for binding to a cytosine, uracil, or thymine base in the opposite strand comprising a boronated C and U or T modified binding base having a boron-containing substituent selected from the group consisting of —BH₂CN, —BH3, and —BH₂ COOR, wherein R is C1 to C18 alkyl; phosphoramidate internucleoside linkages such as a 3′ aminophosphoramidate, aminoalkylphosphoramidate, or aminoalkylphosphorthioamidate internucleoside linkage; modified sugar and/or backbone modifications such as a peptide nucleic acid, a peptide nucleic acid mimic, a morpholino nucleic acid, hexose sugar with an amide linkage, cyclohexenyl nucleic acid (CeNA), or an acyclic backbone moiety; a 3′ terminal cap group;

In one embodiment RNAi agents may be delivered directly to cells for example by transfection or may be delivered by use of viral or non-viral vectors capable of infecting or otherwise transfecting target cell. The vectors can thus deliver and express RNAi agents in situ. The RNAi agents may be transcribed as short hairpin RNA (shRNA) precursors from a viral or non-viral vector. After transcription the shRNA are processed by the enzyme Dicer into the appropriate active RNAi agents, such as siRNA. Viral vectors typically exploit the tissue specific targeting properties of viruses and once appropriately targeted rely upon the endogenous cellular machinery to generate sufficient levels of the RNAi agents to achieve a therapeutically effective dose.

In another embodiment RNAi agents useful in the present disclosure are DNA-directed RNAi (ddRNAi) agents. ddRNAi agents comprise an expression cassette or ddRNAi expression cassette typically comprising at least one promoter, at least one ddRNAi sequence and at least one terminator in a viral or non-viral vector. RNAi agents useful in the present disclosure can be synthetically or enzymatically produced and purified by any protocol known to those skilled in the art using standard techniques as described in, for example, Sambrook, et al. Molecular Cloning; A Laboratory Manual, 2^{nd} Ed., Cold Spring Harbour Press, Cold Spring Harbour, N.Y. (1989).

Alternatively corresponding dsDNA can be employed, which, once presented intracellularly is converted into dsRNA. Methods for the synthesis of suitable molecules for use in RNAi and for achieving post-transcriptional gene silencing are known to those of skill in the art.

dsRNA for use in the present disclosure is preferably derived from a template such as all or part of the endogenous Midline-1 nucleotide sequence or a cDNA produced from an isolated mRNA for example by reverse transcriptase. The dsRNA may be synthesised in vivo or in vitro for example using manual and/or automated procedures. In vitro synthesis may be chemical or enzymatic, for example using cloned RNA polymerase (e.g., T3, T7, SP6) for transcription of the endogenous DNA or cDNA template, or a mixture of both. In vivo, dsRNA may be produced using recombinant techniques well known in the art. For example, bacteria transformed with an expression vector comprising the DNA template encoding the dsRNA can be used as a source of dsRNA. Alternatively, mammalian cells in which inhibition of Midline-1 expression is desired may be transformed with an expression vector for example by infection with a recombinant virus carrying a expression vector comprising the DNA template encoding the dsRNA. Bidirectional transcription of one or more copies of the template may occur by the action of endogenous RNA polymerase of the transformed cell. Alternatively a recombinant RNA polymerase (e.g., T3, T7, SP6) encoded by the expression vector or a second expression vector may be utilised.

Chemically synthesised dsRNA may be purified prior to introduction into the cell. Purification may be by extraction with a solvent (such as phenol/chloroform), precipitation (for example in ethanol), electrophoresis, chromatography, or combinations thereof. However, as purification may reduce the yield of dsRNA in some embodiments purification may be minimal or not carried out at all. The dsRNA may be dried or dissolved in an aqueous solution which may contain buffers or salts to promote annealing, and/or stabilisation of the RNA strands.

dsRNA useful in the present disclosure may contains one or more modified bases to increase stability. For example the phosphodiester linkages of the dsRNA may be modified to include at least one of a nitrogen or sulphur heteroatom. The dsRNA may comprise inosine, or modified bases, such as tritylated bases. It will be appreciated that a variety of modifications have been made to RNA that serve many useful purposes known to those of skill in the art.

The double-stranded structure of dsRNA may be formed by a single self-complementary RNA strand or two separate complementary RNA strands. RNA duplex formation may be initiated either inside or outside the mammalian cell.

The dsRNA sequence of the dsRNA is typically substantially identical to at least a part of the Midline-1. Preferably there is 100% sequence identity between the dsRNA and at least part of the Midline-1 gene. However, dsRNA having 70%, 80% or greater than 90% or 95% sequence identity to Midline-1 may be used in the present disclosure. Thus sequence variations that might be expected due to genetic mutation, strain polymorphism, or evolutionary divergence can be tolerated. The optimum length of the dsRNA may vary and the duplex region of the dsRNA may be at least 15, 25, 50, 100, 200, 300, 400 or more bases long.

Inhibition of the expression of Midline-1 can be verified by observing or detecting an absence or observable decrease in the level of Midline-1 protein, for example using a specific antibody; and/or Midline-1 mRNA such as by hybridisation studies. In the context of a treatment of a condition such as asthma, verification of inhibition of the expression of Midline-1 may be observed by a change in the disease condition of a subject, such as a reduction in symptoms, a change in the disease state and the like. Preferably, the inhibition is specific, i.e. the expression of Midline-1 is inhibited without manifest effects on the other genes of the cell.

Mammalian cells can respond to extracellular dsRNA and thus a dsRNA import mechanism may exist (Asher et al). Accordingly dsRNA may be administered extracellularly for example by aerosol to the airway tissue, into the circulation, or orally.

Antisense oligonucleotides may be prepared by methods well known to those of skill in the art. Typically oligonucleotides will be chemically synthesized on automated synthesizers. Those skilled in the art will readily appreciate that antisense oligonucleotides need not display 100% sequence complementarity to the target sequence. One or more base changes may be made such that less than 100% complementarity exists whilst the oligonucleotide retains specificity for its target and retains antagonistic activity against this target. Suitable antisense oligonucleotides include morpholinos where nucleotides comprise morpholine rings instead of deoxyribose or ribose rings and are linked via phosphorodiamidate groups rather than phosphates.

The antisense nucleotide sequences may have a length of about 20 nucleotides, but may range in length from about 20 to about 200 nucleotides, or may be the entire length of the Midline-1 gene. The skilled person can select an appropriate target and an appropriate length of antisense nucleic acid in order to have the desired therapeutic effect by standard procedures known to the art, and as described, for example, in Methods in Enzymology, Antisense Technology, Parts A and B (Volumes 313 and 314) (M. Phillips, ed., Academic Press, 1999).

A further means of inhibiting the expression or activity of Midline-1 may involve introducing catalytic antisense nucleic acid constructs, such as ribozymes, which are capable of cleaving Midline-1 mRNA transcripts. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave Midline-1 mRNA sequences can be achieved by techniques well known to those in the art (for example Lieber and Strauss, (1995) Mol. Cell. Biol. 15:540-551, the disclosure of which is incorporated herein by reference).

Contemplated herein are engineered ribozymes for instance the hammerhead motif ribozyme, that specifically catalyse endonucleolytic cleavage of Midline-1 mRNA. Specific ribozyme cleavage sites within any the Midline-1 mRNA are initially identified by locating ribozyme cleavage sites, which include the sequences, GUA, GUU and GUC. Once located short RNA sequences of between 15 and 20 ribonucleotides corresponding to the region of the Midline-1 mRNA containing the cleavage site may be evaluated for predicted structural features such as secondary structure that may render the oligonucleotide sequence unsuitable. Both anti-sense RNA and DNA molecules and ribozymes may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors, which incorporate suitable RNA polymerase promoters such as the T7 or SP6 polymerase promoters. Methods of making ribozymes are well known in the art. Construction of suitable vectors containing the desired ribozyme sequences and control sequences typically employs standard ligation and restriction techniques, which are well known in the art (see Sambrook et al.).

If desired, agents for use in accordance with the present disclosure may be fused to other compounds, including peptides, polypeptides or other proteinaceous or non-proteinaceous molecules. For example, agents may be fused to molecules to facilitate localisation to the airway tissue.

Screening for suitable modulatory agents for use in accordance with the present disclosure may be achieved by any one of several suitable methods including, but in no way limited to, contacting a cell comprising a gene encoding Midline-1 or a functional equivalent or derivative thereof with an agent and screening for the modulation of Midline-1 protein production or functional activity, modulation of the expression of a nucleic acid molecule encoding Midline-1 or modulation of the activity or expression of a downstream Midline-1 cellular target. Detecting such modulation can be achieved utilising techniques such as Western blotting, electrophoretic mobility shift assays, mass spectroscopy, and/or the readout of reporters such as luciferases, chloramphenicol acetyltransferase (CAT) and the like. Examples of screening methods suitable for screening for substances which modulate Midline-include a cell based assay carried out by transfecting cells with nucleic acid encoding Midline-1 or using a cell line which endogenously expresses Midline-1 and determining the effect that candidate substances have on Midline-1 expression and/or activity. The cells may be co-transfected with a reporter construct designed to produce an easily detectbale protein (e.g. GFP, luciferase, CAT and the like) in response to Midline-1 expression. A candidate substance or group of candidate substances are incubated with the cells and the level and/or activity of Midline-1 is subsequently assessed. The screening methods may be carried out in a multiplex assay format in which a solid phase is employed on which a plurality of cells expressing Midline-1 are immobilised in separate containers (e.g. in a multi-well plate). Each candidate substance or each group of candidate substances may be applied to different wells.

### Pharmaceutical Compositions

Agents may typically be administered in accordance with the present disclosure in the form of pharmaceutical compositions, which compositions typically comprise one or more pharmaceutically acceptable carriers, excipients or diluents. Such compositions may be administered in any convenient or suitable route such as by parenteral, oral, nasal or topical routes. In particular embodiments administration is via the respiratory tract, including, for example, oral, intranasal, sublingual, intrapulmonary, or intratracheal administration. Administration via the respiratory tract may be via any suitable form (solid, liquid or gaseous dosage form), e.g. inhalation or insufflation of powders or aerosols using, for example, a pressurized metered dose inhaler, nebulizer or vaporizer, intranasal administration of a dry powder, aerosol or nasal spray, or oral administration of any suitable solid or liquid dosage form as is well known to those skilled in the art. The skilled addressee will appreciate that any suitable dosage form and route of administration may be employed in accordance with the present disclosure.

In circumstances where it is required that appropriate concentrations of the agent are delivered directly to the site in the body to be treated, administration may be regional rather than systemic. Regional administration provides the capability of delivering very high local concentrations of the agent to the required site and thus is suitable for achieving the desired therapeutic or preventative effect whilst avoiding exposure of other organs of the body to the agent and thereby potentially reducing side effects.

It will be understood that the specific dose level of a composition of the disclosure and the most suitable and effective route of administration for any particular individual will depend upon a variety of factors including, for example, the activity of the specific agents employed, the age, body weight, general health and diet of the individual to be treated, the nature and extent of any condition suffered by the individual, the time of administration, rate of excretion, and combination with any other treatment or therapy. Single or multiple administrations can be carried out with dose levels and pattern being selected by the treating physician. A broad range of doses may be applicable. In some embodiments, an effective amount for a human subject lies in the range of about 0.1 ng/kg body weight/dose to 1 g/kg body weight/dose. In some embodiments, the range is about 1 μg to 1 g, about 1 mg to 1 g, 1 mg to 500 mg, 1 mg to 250 mg, 1 mg to 50 mg, or 1 μg to 1 mg/kg body weight/dose. Dosage regimens may be adjusted to provide the optimum therapeutic response. For example, several divided doses may be administered daily, weekly, monthly or other suitable time intervals or the dose may be proportionally reduced as indicated by the exigencies of the situation.

Compositions may be conveniently presented in unit dosage form and prepared by any of the methods well known in the art of pharmacy. The method may include the step of bringing the components of the composition into association with a carrier, e.g. a liquid carrier or finely divided solid carrier, which constitutes one or more accessory ingredients.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The agent may be suitably protected to facilitate oral administration, for example, using an inert diluent or with an assimilable edible carrier, or by enclosing in a hard or soft shell gelatin capsule, or being compressed into tablets, or being incorporated directly with the food in the diet. For oral therapeutic administration, the agent may be incorporated with excipients and used in the form of ingestible tablets, buccal tablets, troches, capsules, elixirs, suspensions, syrups, wafers, and the like.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinyl pyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleatestearate or -laurate, polyoxyethylene sorbitan mono- or di-oleatestearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Suitable topical formulations may comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90 C-100 C for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturizer such as glycerol, or oil such as castor oil or arachis oil.

Creams, ointments or pastes are typically semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the agent in the required amount in the appropriate solvent with various of the other ingredients enumerated above, as required, followed by filter sterilisation. Generally, dispersions are prepared by incorporating the agent into a sterile vehicle which contains the basic dispersion medium and any other required ingredients. In the case of sterile powders for the preparation of sterile injectable solutions, the preferred methods of preparation are vacuum drying and the freeze-drying technique which yield a powder of the agent plus any additional desired ingredient from previously sterile-filtered solution thereof.

The present disclosure also contemplates combination therapies, wherein agents the subject of the present disclosure are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments disclosed herein. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of agent. Administration may be in any order.

Diagnosis

An aspect of the disclosure provides a method for diagnosing a condition associated with airway inflammation and/or airway tissue remodelling, or susceptibility or predisposition thereto, in a subject, the method comprising determining the level of Midline-1 in a biological sample from the subject.

It should be understood that the biological sample which is screened in accordance with this aspect of the present disclosure may be any suitable sample which would be indicative of the Midline-1 level of the airway tissue. The sample may be a biopsy sample of the airway tissue or it may be some other form of sample such as blood (serum), induced sputum, exhaled breath condensate, or lavage sample.

Although the preferred method is to screen for an increase in Midline-1 levels in order to diagnose susceptibility to a condition, the detection of a decrease in the level of this molecule may be desired under certain circumstances, for example, to monitor for improvement or responsiveness to a therapeutic or prophylactic treatment regimen.

The present disclosure should therefore be understood to extend to screening for decreases in Midline-1 levels. Those skilled in the art will appreciate that in the context of this type of screening protocol one may seek to analyse a screening result relative to an earlier obtained result rather than only relative to normal levels.

Methods of screening for levels of Midline-1 can be achieved by any suitable method which would be well known to those skilled in the art. In this regard, it should be understood that reference to screening for the level of protein and/or gene expression “in a subject” is intended as a reference to the use of any suitable technique which will provide information in relation to the level of expression of Midline-1 in the relevant tissue of the subject. Accordingly, these screening techniques include both in vivo screening techniques, as well as in vitro techniques which are applied to a biological sample extracted from the subject. Such in vitro techniques are likely to be preferred due to their significantly more simplistic and routine nature.

Since embodiments of the present disclosure are predicated, in part, on screening for changes in the level of Midline-1 such changes can in fact be screened for at the protein level or at the nucleic acid level, such as by screening for increases in the level of Midline-1 mRNA transcripts. Those skilled in the art will be able to determine the most appropriate means of analysis in any given situation.

Without limiting the scope of the disclosure in any way, suitable methods of identification of Midline-1 levels include in vivo molecular imaging (e.g. Weissleder, R et al., Nature Medicine, 6:351-355, 2000), fluorescent in situ hybridisation (FISH), quantitiative reverse transcriptase PCR (QRTPCR), flow cytometry, and immunoassay such as enzyme-linked immunosorbent assay (ELISA). Suitable immunoassay techniques are described, for example, in U.S. Pat. Nos. 4,016,043, 4,424,279 and 4,018,653. These include both single-site and two-site assays of the non-competitive types, as well as traditional competitive binding assays. These assays also include direct binding of a labelled antigen-binding molecule to a target antigen. Techniques and protocols for employing such methods are well known to those skilled in the art. Any suitable technique may be utilised to detect Midline-1 or its encoding nucleic acid molecule. The nature of the technique which is selected for use will largely determine the type of biological sample which is required for analysis. Such determinations are well within the scope of the person of skill in the art. Typical samples which one may seek to analyse are biopsy samples of the airways.

The present disclosure also provides kits suitable for use in accordance with the methods of the disclosure. Such kits include for example diagnostic kits for assaying biological samples, comprising an agent for detecting Midline-1 or encoding nucleic acid molecules, and reagents useful for facilitating the detection by the agent(s). Further means may also be included, for example, to receive a biological sample. The agent(s) may be any suitable detecting molecule. Kits according to the present disclosure may also include other components required to conduct the methods of the disclosure, such as buffers and/or diluents. The kits typically include containers for housing the various components and instructions for using the kit components in the methods of the present disclosure.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure is further described by reference to the following non-limiting examples.

### EXAMPLES

The following general methods were employed in the studies described herein below.

Mice. WT, Tnfsf10^{-/-}, Tlr4^{-/-}, Myd88^{-/-}, and Stat6^{-/-} BALB/c mice (6-14 weeks) were obtained from the Special Pathogen Free Facility of the University of Newcastle. The animal ethics committee of the University of Newcastle, Australia approved all experiments.

Induction of Allergic Airways Disease.

Mice were sensitized and challenged by exposing them intranasally to house dust mite (HDM) (50 mcg daily at day 0, 1, and 2 followed by 5×5 mcg daily from day 14 to day 17 delivered in 50 μl of sterile saline). The single dose of HDM employed in initial studies was 50 mcg/50 μl of sterile saline. Non-sensitized mice received sterile saline only.

AHR Measurement.

AHR was assessed invasively in separate groups of anesthetized mice by measurement of total lung resistance and dynamic compliance. Percentage increase over baseline (water) in response to nebulised methacholine was calculated.

Isolation of mRNA.

Total RNA was isolated with mirVana m/miRNA Isolation kit (Ambion) from lower airway tissue that was separated by blunt dissection allowing effective separation of the airway wall from the parenchyma (Mattes, 2009).

Quantitative RT-PCR.

quantitative RT-PCR (qRT-PCR) was performed with SYBR® Green (Invitrogen) using an Eppendorf Realplex2. CT (threshold cycle) quantification was performed using Eppendorf Realplex analysis software 2.2. Mouse mRNA expression was normalised to HPRT, human mRNA to GAPDH. Fold change was calculated using delta delta CT values relative to control groups. The primers sequences used are shown in table 1.

TABLE 1
RT-PCR Primers

| | | SED |
|---|---|---|
| Primer | Sequence (5′ to 3′) | ID NO. |
| GAPDH Human Forward | ACAGTCAGCCGCATCTTCTTTTG | 4 |
| GAPDH Human Reverse | CCAATACGACCAAATCCGTTGAC | 5 |
| Mid-1 Human Forward | GTACACCATATTCACCGACAAGC | 6 |
| Mid-1 Human Reverse | AGTGGTTCTGCTTGATGTTGGGTA | 7 |
| HPRT Mouse Forward | AGGCCACACTTTGTTGGATTTG | 8 |
| HPRT Mouse Reverse | CAACTTGCGCTCATCTTAGGCTTT | 9 |
| Mid-1 Mouse Forward | CACTCGCTGAAGGAAAATGACCA | 10 |
| Mid-1 Mouse Reverse | AATCCAAGGCAAAAGTGTCAAACG | 11 |

Airway Morphology Studies.

Lung tissue was stained, cells identified by morphological criteria, and quantitated by counting ten high power fields (HPFs) in each slide.

Cytokine Analysis.

Peribronchial lymph node cells were excised, filtered, and cultured in the presence or absence of 50 mcg/ml HDM (optimal concentration) for 6 days. IL-13, IL-5, and IFN-γ were determined by ELISA (BD Biosciences Pharmingen).

PP2a Activity Assay.

PP2a activity was measured by R&D Systems PP2A DuoSet IC activity assay kit according to the manufacturer's instructions. This was performed on homogenized mouse lungs and harvested BEAS-2B cells.

siRNA.

siRNAs with no similarities to other sequences were obtained from Ambion (Applied Biosystems). Sequence of the antisense strand of siRNA-Midline-1 was: 5′-UUAGGUAAUCCAGACAUUCta-3′ (SEQ ID NO: 3). Nonsense siRNA (chosen to have an equivalent CG content) with no similarities to other sequence was obtained from Ambion (Option 2). 3.75 nmol siRNA/25 μl of sterile saline was administered intranasally at day 13 (after HDM sensitization and 24 hrs before first HDM challenge) and then very second day until mice were sacrificed.

Immunofluorescent Detection.

Formalin-preserved lung sections were fixed and incubated with either anti-Midline-1 antibody followed by a secondary PE-conjugated antibody (Santa Cruz Biotechnology), or an AlexaFluor®488-conjugated antibody against phosphorylated-p38 (Cell Signalling Technology). Nuclei were counterstained with DAPI (Sigma). Microscopic analysis was performed with an Olympus BX51 UV microscope using DP Controller 3.1.1.267 software (Olympus).

Statistics.

The significance of differences between groups was analyzed using Student's t-test, Mann-Whitney test, or two-way ANOVA as appropriate.

### Example 1

### TRAIL Regulates HDM-Induced Allergic Airways Disease, Midline-1 and PP2A Activity

The present inventors have recently identified Tumor necrosis factor (TNF)-related apoptosis-inducing ligand (TRAIL), which is a member of the TNF superfamily of cytokines, as innate immune signal released by the respiratory epithelium upon allergen exposure (Weckmann et al., 2007). TRAIL was essential for the expression of all hallmark features of ovalbumin-induced allergic airways disease (AAD) by promoting CCL20-mediated recruitment of T cells and dendritic cells in a STAT6-dependent manner.

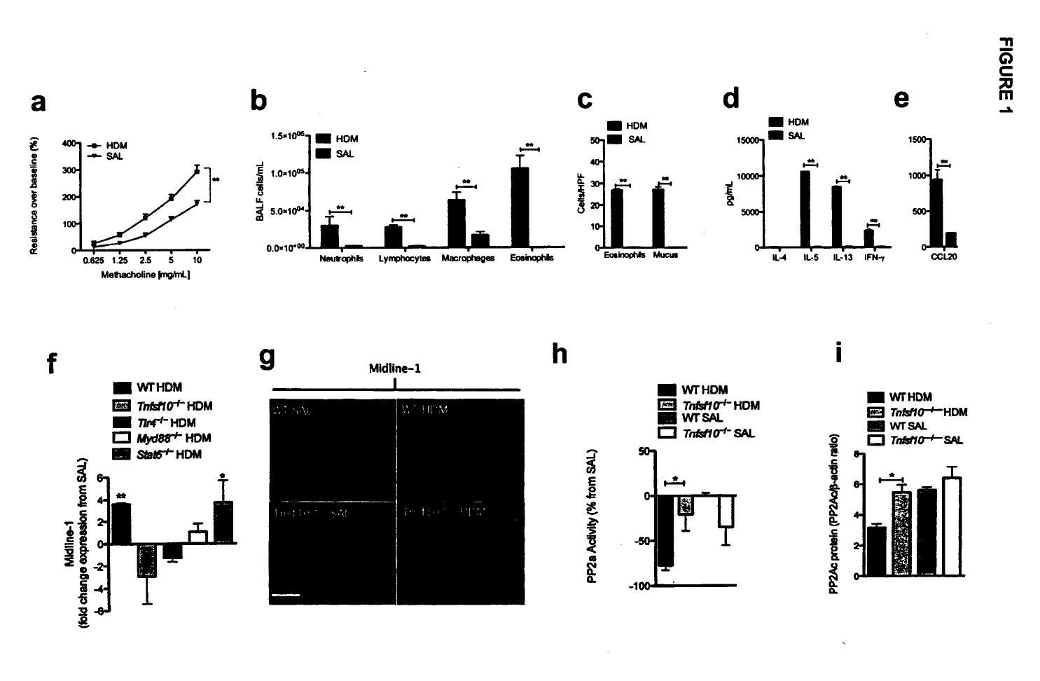

A role for TRAIL in TLR4-dependent house dust mite-induced allergic airways disease is demonstrated herein, including airways hyperresponsiveness (FIG. 1a-), inflammatory cell accumulation in bronchoalveolar lavage fluid (FIG. 1b), airways inflammation and mucus production (FIG. 1c), release of Th2 cytokines from in-vitro allergen-stimulated peribronchial lymphnode cells (FIG. 1d), and CCL20 release (FIG. 1e). To identify novel signaling pathways in these models airway tissue from lung parenchyma was blunt dissected and transcripts determined that were differentially expressed in the airway tissue of allergic wild type versus wild type mice employing genome-wide gene array analysis and confirmed by qPCR. The inventors found that the microtubule-associated E3 ubiquitin ligase Midline-1 was upregulated in allergic as compared to non-allergic WT mice. However, Midline-1 was not upregulated in the respiratory epithelium of house dust mite allergic Tnsfs10^{-/-} mice (FIG. 1f). Crude house dust mite extract is sensed by TLR4 and this signal, like TRAIL, is required for the development of allergic airways disease. Notably, mice deficient for TLR4 (Tlr4^{-/-}) also failed to upregulate Midline-1 in response to allergen exposure (FIG. 1f).

The intracellular TLR4 receptor domain (Toll/Interleukin-1 receptor (TIR) domain) binds to the adapter molecule MyD88 for signal transduction. Reduced Midline-1 expression was also found in allergen-exposed Myd88^{-/-} as compared to wild type mice (FIG. 1f). In contrast however, allergen-sensitized and challenged Stat6^{-/-} mice showed increased Midline-1 expression (FIG. 1f) although this mouse strain was, like Tlr4^{-/-} and Myd88 mice, fully protected from the development of allergic airways disease. The data suggests that TRAIL activated by TLR4/MyD88-dependent allergen sensing in airway epithelial cells promotes Midline-1 expression upstream of STAT6-dependent IL-13 production, and it is therefore suggested that Midline-1 links TLR4 and TRAIL signaling to hallmark features of allergic airways diseases.

Midline-1 is required for the ubiquitin-specific modification and proteasomal degradation of the catalytic subunit of protein phosphatase (PP) 2A (PP2Ac) via interaction with a regulatory subunit of PP2A, the α4 subunit. In accordance, PP2A activity and PP2Ac protein expression were suppressed in allergic WT mice in vivo while Midline-1 expression was increased, but each remained unchanged in allergic Tnfsf10^{-/-} as compared to non-allergic wild type mice (FIGS. 1h and i). The PP2A holoenzyme is composed of three subunits but only the PP2A-B subunit is highly variable and confers substrate specificity while PP2A-A and PP2Ac are the highly conserved scaffolding and catalytic proportions, respectively. PP2A is the most abundantly expressed protein phosphatase and regulates protein function by dephosphorylating transcriptional factors such as the MAPK kinase (MAPKK) MKK3/6, which is critically involved in limiting p38 MAPK activation. Notably, the p38 MAPK signalling pathway is activated in the airway wall of severe but not mild asthmatics and promotes airways hyperresponsiveness, inflammation, mucus hypersecretion, subepithelial fibrosis, smooth muscle hypertrophy and cytokine/chemokine release (for example Liu et al., 2008).

### Example 2

### Midline-1 Inhibition Abolishes Airways Hyperreactivity, Reduces Airway Inflammation and Increases PP2a Activity

To determine the role of Midline-1 in allergic airways disease, expression of Midline-1 was directly inhibited. Specifically, allergic mice were treated with a commercially available (Ambion) in-vitro validated small interfering (si)RNA directed against Midline-1 (SEQ ID NO:3) (MID1 siRNA) or a scrambled nonsense siRNA (control) following previously established protocols. BALB/c mice were anesthetised with isoflurane before being sensitised with the intranasal administration of either 50 μg House Dust Mite antigen (HDM) in 50 μl of sterile saline or 50 μl of sterile saline in control groups. This sensitisation was repeated at days 1 and 2 following initial sensitisation. On day 14 following initial sensitisation the mice were again anesthetised with isoflurane before receiving intranasal challenge with 5 μg of HDM in 500 of sterile water. This challenge dosage was repeated daily from day 14 through 17. Mice received intranasal administration of maked Mid-1 siRNA or scramble control in 25 μl of saline under isoflurane anaesthesia on days 13, 15 and 17, through the period of HDM challenge. On day 18, 24 hrs after the final HDM challenge, the mice were sacrificed with an excess dose of sodium pentobarbitone administered intraperitoneally (200 μl total volume).

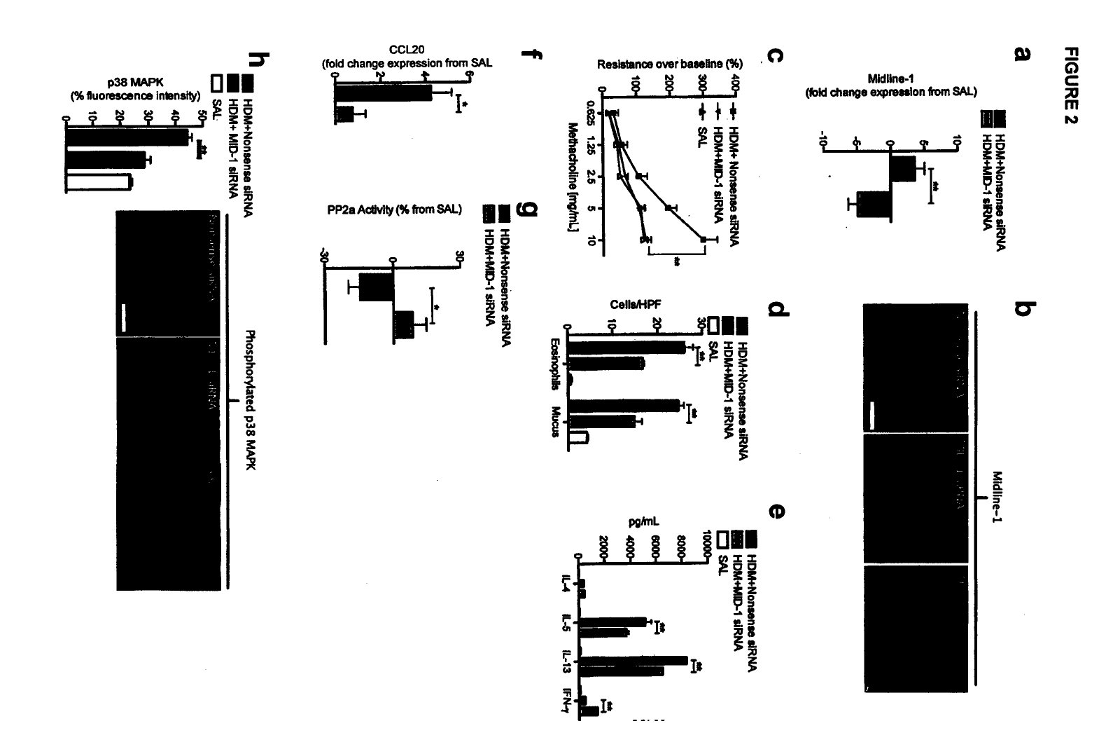

Inhibition of Midline-1 during allergen challenge in sensitized wild type mice in the airway wall (FIG. 2a and b) abolished airways hyperresponsiveness (FIG. 2c), suppressed airways inflammation and mucus production (FIG. 2d), and reduced Th2 cytokine release in allergen-stimulated peribronchial lymphnode cultures (FIG. 2e), and CCL20 expression in blunt-dissected airway wall samples (FIG. 20. Importantly this was inversely correlated to PP2A activity (FIG. 2g) and levels of phosphorylated p38 MAPK (FIG. 2h). Therefore Midline-1 expression downstream of the TLR4-induced TRAIL signalling cascades is essential in the development of allergic airways diseases and directly linked to PP2A activation levels.

### Example 3

### PP2A Activation Abolishes Airways Hyperreactivity and Reduces Airway Inflammation and Th2 Cytokine Release

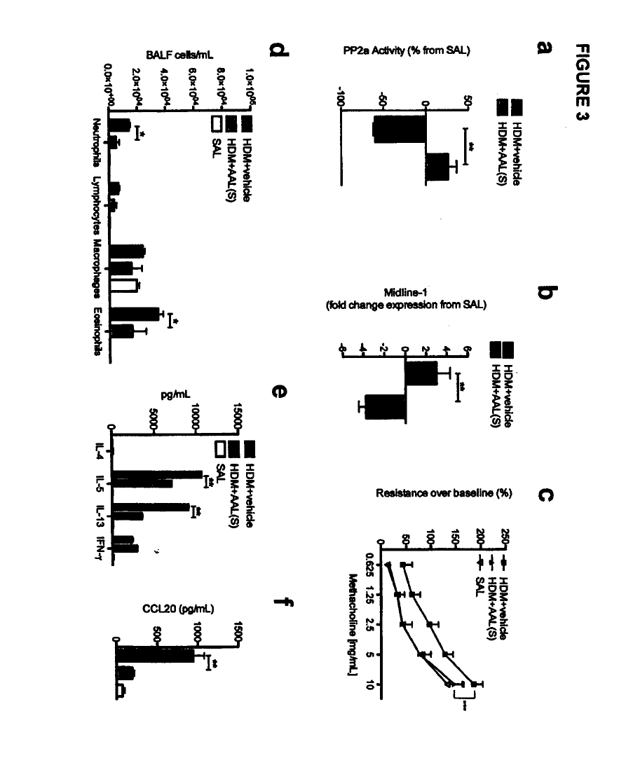

To confirm a causal relation between PP2A levels in the lung and asthma pathogenesis allergic WT mice were treated with a non-phosphorylatable FTY720 analogue, 2-amino-4-(4-heptyloyphenol)-2-methylbutanol (AAL_{(S)}), that reactivates PP2A in the lungs (FIG. 3a) but does not bind to sphingosine-1-phosphate receptors like FTY720 (Don et al., 2007) because it cannot be phosphorylated by sphingosine kinase 2 (and therefore does not cause lymphopenia). Interestingly PP2A activity was inversely correlated with Midline-1 expression (FIG. 3b). Here evidence is provided for an interaction between PP2A and Midline-1 on a transcriptional or post-transcriptional level. Notably, daily intranasal treatment with AAL_{(S)} during repeated house dust mite challenges to sensitized mice precluded the development of airways hyperresponsiveness (FIG. 3c), airways inflammation (FIG. 3d), and reduced Th2 cytokine (FIG. 3e) and CCL20 release (FIG. 31). This suggests that raising PP2A activity in the lungs downregulates Midline-1 activity, prevents AHR and attenuates allergic lung inflammation.

### Example 4

### Midline-1 Inhibition in Rhinovirus-Associated Airways Inflammation and Asthma Exacerbation

No effective treatment for rhinovirus-associated asthma exacerbations is currently available. To investigate the therapeutic potential of Midline-1 inhibition for virus-associated airways inflammation and asthma exacerbation, a mouse model of rhinovirus-associated airways inflammation and obstruction was employed (Bartlett, 2008).

BALB/c mice were anesthetised with isoflurane before being sensitised with the intranasal administration of either 50 μg House Dust Mite antigen (HDM) in 50 μl of sterile saline or 50 μl of sterile saline in control groups. This sensitisation is repeated at days 1 and 2 following initial sensitisation. On day 14 following initial sensitisation the mice are again anesthetised with isoflurane before receiving intranasal challenge with 5 μg of HDM in 50 μl of sterile water. This challenge was repeated daily from day 14 through 17. Mice received intranasal administration of naked Mid-1 siRNA or scramble control in 25 μl of saline under isoflurane anaesthesia on days 13, 15 and 17, through the period of HDM challenge. On day 18, 24 hrs after the final HDM challenge, the mice are infected with Rhinovirus 1b through intranasal inoculation with 50 μl or 1×10⁷ (TCID50) of virus whilst under isoflurane induced anaesthesia. On day 19, 24 hrs after viral inoculation mice were sacrificed with an excess dose of sodium pentobarbitone administered intraperitoneally (200 μl total volume).

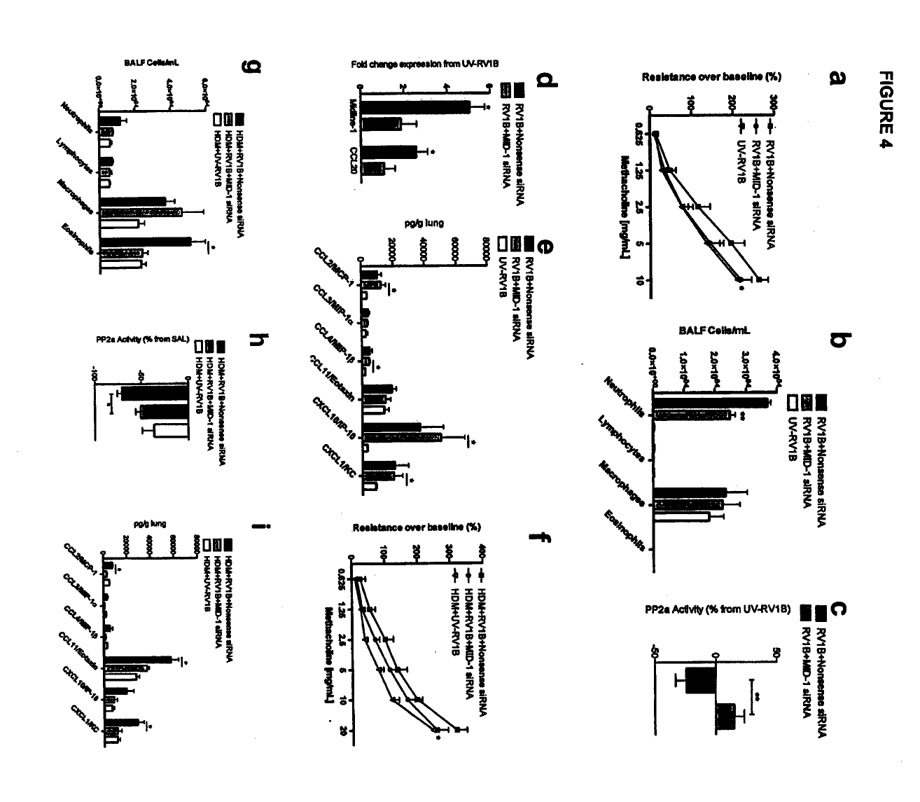

First non-allergic mice were challenged with rhinovirus (RV1B, minor group). 24 hrs after RV1B exposure, naïve mice treated with the control siRNA developed a neutrophil-dominated lung inflammation and airways hyperreactivity, which coincided with reduced PP2A activity and elevated Midline-1 and CCL20 expression (FIG. 4a to d). RV1B infection also led to the upregulation of a range of chemokines that promote lung inflammation (FIG. 4e). Inhibition of Midline-1 24 hrs before RV1B exposure fully protected mice from RV1B-induced AHR, reduced neutrophilic airways inflammation, and CCL20 expression (FIG. 4a to d) but did not affect other chemokines (FIG. 4e), virus replication or increased anti-viral interferon responses in non-allergic mice.

Next, allergic mice were infected with RV 1B 24 hrs after their final house dust mite allergen challenge. BALB/c Mice received intranasal administration of naked Mid-1 siRNA or scramble control in 25 μl of saline under isoflurane anaesthesia on day 0. On day 1, mice were infected with Rhinovirus 1b through intranasal inoculation with 50 μl of 1×10′ (TCID50) of virus whilst under isoflurane induced anaesthesia. On day 2, 24 hrs after viral inoculation mice were sacrificed with an excess does of sodium pentobarbitone administered intraperitoneally (200 μl total volume).

Importantly treatment with a siRNA directed against Midline-1 24 hrs before virus challenge ameliorated rhinovirus-associated exacerbation of eosinophilic inflammation and AHR as compared to those allergic mice treated with a nonsense siRNA (FIG. 4f and g). Midline-1 inhibition also raised PP2A activity (FIG. 4h) and impaired rhinovirus-associated chemokine release in lung homogenates (FIG. 4i) and house dust mite-specific IL-5 (Mean [SE]: Nonsense siRNA 8.6 ng/ml [0.6] versus MID1-siRNA 3.2 ng/ml [0.2]; p<0.01) but not IL-13 production by peribronchial lymphnode cells (data not shown). As observed in naive mice inhibition of Midline-1 did not affect RV 1B replication or stimulate anti-viral IFN responses (data not shown). Therefore inhibition of Midline-1 just before virus challenge protects mice from rhinovirus-associated AHR independent of affecting viral replication. Thus, blocking Midline-1 in the airways represents a novel therapeutic strategy for preventing rhinovirus-associated exacerbations.

### Example 5

### TRAIL, Midline-1 and PP2A Upregulation After Rhinovirus Infection

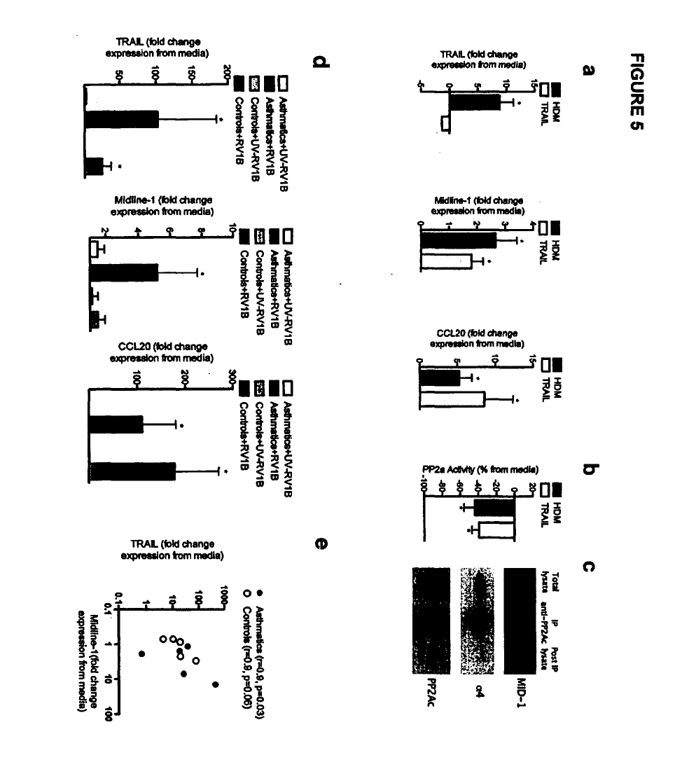

To confirm a direct link between TRAIL, Midline-1, and PP2A activity, immortalized human airway epithelial cells (BEAS-2B) were stimulated with TRAIL or house dust mite extract for 24 hrs. As expected an increase in TRAIL expression was observed after stimulation with house dust mite extract but not after TRAIL stimulation (FIG. 5a). Midline-1 and CCL20 (FIG. 5a) were also expressed upon house dust mite and TRAIL exposure, but PP2A activity was suppressed in these cells (FIG. 5b). BEAS-B2 cells were lysed and western blotted for Midline-1, the alpha4 subunit of PP2A, and PP2Ac (FIG. 5c: lane one). Immunoprecipitation for the PP2Ac subunit were performed and both Midline-1 and alpha4 were detected in the PP2Ac immunoprecipitant (FIG. 5c) confirming the direct interaction of Midline-1, PP2Ac, and the alpha4 subunit of PP2A.

Primary airway epithelial cells collected from asthmatics and healthy subjects were exposed to infective or UV-inactivated RV1B. Upregulation of Midline-1, CCL-20, and TRAIL was observed 24 hrs after rhinovirus infection (FIG. 5d). Notably, Midline-1 was directly correlated with TRAIL expression (FIG. 5e). In independent experiments, healthy and asthmatic primary airway epithelial cells were infected with RV1B for 6 hrs only and this confirmed a strong correlation between TRAIL and Midline-1 expression (n=18; r=18; p<0.01). Thus, rhinovirus induces Midline-1, which is an essential molecule in the establishment of airways inflammation, obstruction, and asthma exacerbation.

### Example 6

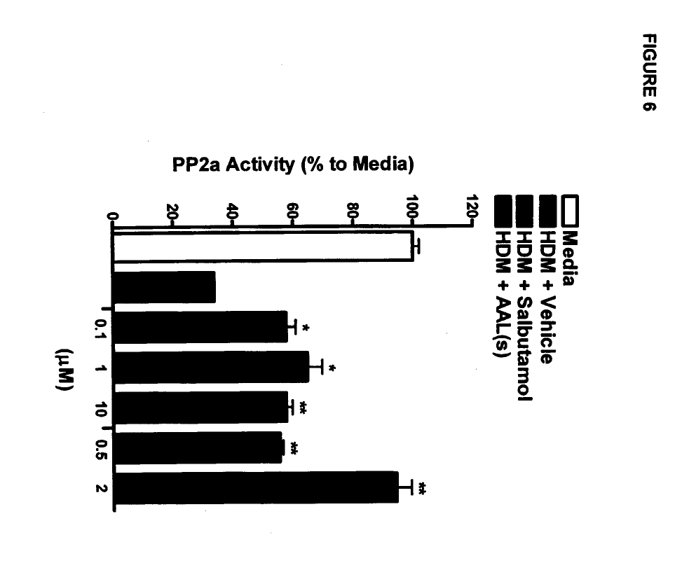

Activation of PP2A The inventors have examined the ability of known PP2A agonists to influence PP2A activity in human bronchial epithelial cells stimulated with crude house dust mite extract. Immortalised human bronchial epithelial cells (BEAS-2B) were seeded at 1×10⁵ cells/well in 12-well tissue culture plates and allowed to reach 80% confluency. Cells were then treated with either salbutamol, a mitoxantrone analogue, or AAL(s), at a range of tolerable concentrations (0.1, 1 and 10 μM for salbutamol; 1, 5 and 25 μg/ml for the nitoxantrone analaogue; and 0.5 and 2 μM for AAL(s)). Simultaneously, cells were stimulated with crude House Dust Mite (HDM) extract and left for 24 hours. Cells were then collected, washed, lysed, and PP2A activity assayed (R&D Systems), measuring phosphatase activity when fed a synthetic substrate. PP2A activity was reduced upon HDM stimulation compared to the non-stimulated media only group (representing physiologically normal activity levels). Both salbutamol and the mitoxantrone analogue were able to restore PP2A activity to a significant level (see FIG. 6). AAL(s) fully restored PP2A activity in a dose dependent manner.

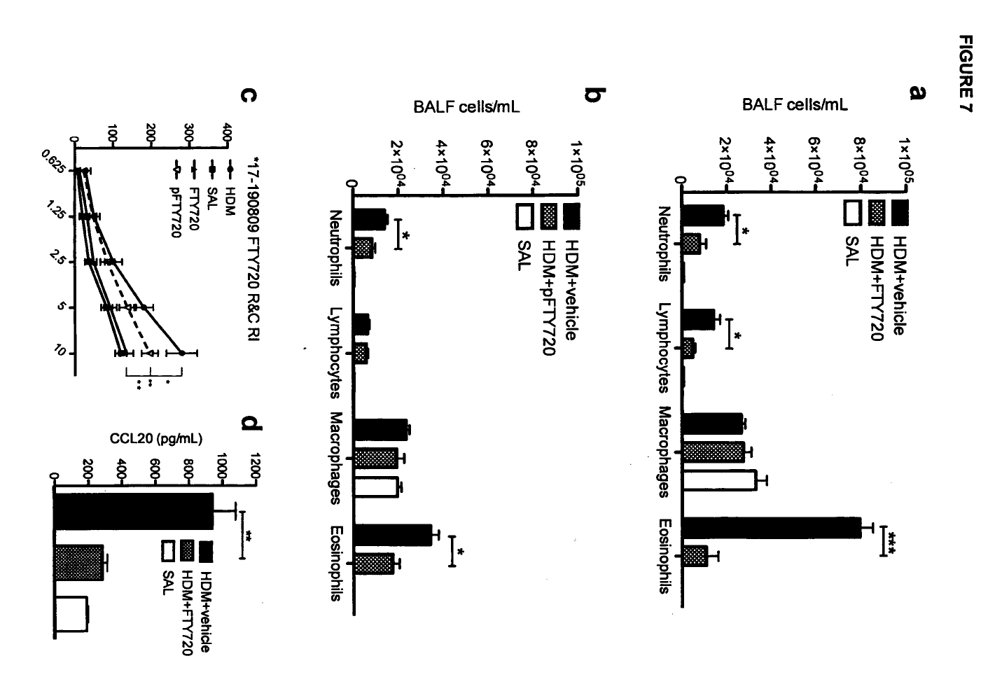

The effect of FTY720 (a known PP2A agonist that signals via sphingosine-dependent and non sphingosine-dependent pathways) and pFTY720 (a synthetically phosphorylated form of FTY720 only able to signal through sphingosine pathways) in the inventors' house dust mite model of allergic airways disease (see examples above) was then determined. The data is shown in FIG. 7. Following FTY720 treatment neutrophils, eosinophils, lymphocytes, AHR and CCL20 were reduced to a similar extent to that seen in TRAIL−/− and AAL_{(S)} treated mice. While pFTY720 treatment was only able to illicit a partial reduction in AHR, neutrophillia and eosinophillia suggest that at least the remainder of the FTY720 effect is through its actions upon PP2A.

### Example 7

### The Effect of AAL_{(S)} on Airways Fibrosis

The inventors then determined the ability of AAL_{(S)} to protect against airway fibrosis in two mouse models, a chronic ovalbumin model of allergic airways disease and an acute bleomycin-induced fibrosis model.

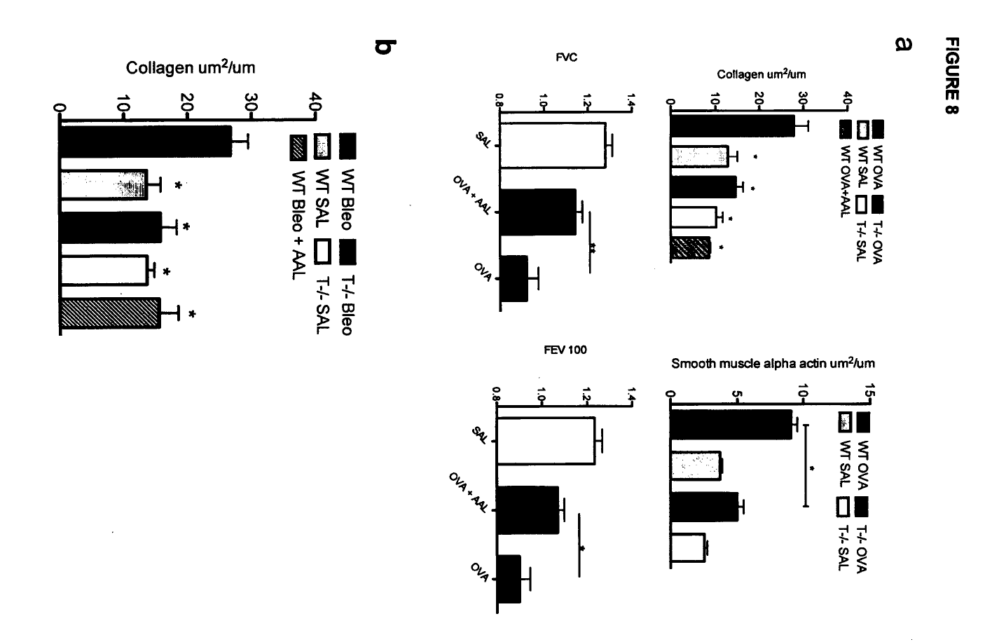

Mice received ovalbumin (OVA)+alumn IP at day 0 and 2 weeks. Mice then received OVA 3× a week for 3 months. AAL treated mice received AAL_{(S)} daily throughout the 3 month challenge period. As shown in FIG. 8a and b, TRAIL-deficient mice and wildtype BALB/c mice who received AAL_{(S)} were protected from airways fibrosis as determined by collagen deposition surrounding the airways (n=1-6). TRAIL −/− mice were also protected from smooth muscle hypertrophy. AAL_{(S)} treated mice also demonstrated improved lung function (forced vital capacity and forced expiratory volume in 100 milliseconds) as determined by a Buxco forced manoeuvres machine.

In the bleomycin model, mice received bleomycin intratracheally and were sacrificed after 23 days. AAL_{(S)} treated mice received AAL_{(S)} daily post bleomycin administration. TRAIL deficient and AAL_{(S)} treated mice were protected from collagen deposition in their airways (n=1-6) (see FIG. 8c).

### REFERENCES

Asher, et al., Nature 223: 715-717 (1969)
Bartlett et al., Nat Med 14, 199-204 (2008)
Don, A. S., et al., J Biol Chem 282, 15833-15842 (2007)
Hammad, H., et al., Nat Med 15, 410-416 (2009)
Kuperman, D., et al., J Exp Med 187, 939-948 (1998)
Liu, W., et al., J Allergy Clin Immunol 121, 893-902 e892 (2008)
Mattes, J., et al., Proc Natl Acad Sci USA 106, 18704-18709 (2009)
Weckmann, M., et al., Nat Med 13, 1308-1315 (2007)

1. A method for treating or preventing an allergic condition, or at least one symptom, manifestation or exacerbation of the condition, the method comprising administering to a subject in need thereof an effective amount of an agent capable of inhibiting the expression and/or activity of Midline-1.

2. The method of claim 1 wherein the condition is associated with airway inflammation and/or airway tissue remodelling.

3-4. (canceled)

5. The method of claim 1 wherein the condition is an allergic airways disease.

6. The method of claim 5 wherein the allergic airways disease is selected from the group consisting of asthma, asthma exacerbations, symptoms of asthma, chronic obstructive pulmonary disease (COPD), pulmonary fibrosis, cystic fibrosis, and a wheezing illness.

7. The method of claim 6 wherein the asthma exacerbations are rhinovirus-associated exacerbations.

8. The method of claim 1 wherein the administration of the agent inhibits, reduces or prevents the establishment of airways hyperresponsiveness, suppresses or inhibits airways inflammation or mucous production, reduces the release of cytokines, inhibits collagen deposition in airway cells and/or inhibits or prevents airways fibrosis.

9. The method of claim 1 wherein the inhibition of expression and/or activity of Midline-1 occurs in one or more abnormal cells implicated in the condition.

10. The method of claim 9 wherein the cells are airway epithelial cells.

11. The method of claim 1 wherein the Midline-1 comprises the amino acid sequence set forth in SEQ ID NO: 1, or is encoded by a nucleotide sequence as set forth in SEQ ID NO: 2.

12. (canceled)

13. The method of claim 1 wherein the agent is a molecule capable of inhibiting or suppressing expression of Midline-1.

14. The method of claim 13 wherein the inhibitor is an antisense construct selected from the group consisting of small interfering RNA (siRNA), catalytic antisense construct and morpholino or other antisense oligonucleotide.

15. (canceled)

16. The method of claim 14 wherein the siRNA comprises the sequence set forth in SEQ ID NO: 3.

17. The method of claim 1 wherein the agent is an inhibitor or antagonist of the activity of the Midline-1 polypeptide.

18. The method of claim 17 wherein the antagonist is an antibody.

19. (canceled)

20. The method of claim 17 wherein the agent is an activator of PP2A.

21. (canceled)

22. The method of claim 20 wherein the agent is a selective activator of PP2A that interacts with PP2A but not sphingosine-1-phosphate receptors.

23. The method of claim 22 wherein the selective activator of PP2A is 2-amino-4-(4-heptyloyphenol)-2-methylbutanol (AAL_{(S)}).

24. The method of claim 1 wherein inhibiting Midline-1 expression or activity comprises modulating the Midline-1 levels in airway tissue of the subject compared to said levels in said tissue before administration of the agent.

25-28. (canceled)

29. A method for diagnosing a condition associated with airway inflammation and/or airway tissue remodelling, or susceptibility or predisposition thereto, in a subject, the method comprising determining the level of Midline-1 in a fluid or in airway tissue or cells of the subject wherein the level of expression of the Midline-1 is indicative of the condition, or a susceptibility or predisposition thereto.

30. The method of claim 29 wherein the fluid is serum or bronchoalveolar lavage fluid.

31. The method of claim 29 wherein the cells are airway epithelial cells.

32-33. (canceled)
